(19) United States
(12) Patent Application Publication
(10) Pub. No.: US 2025/0090429 A1
(43) Pub. Date: Mar. 20, 2025

(54) UV FILTER COMPOSITIONS COMPRISING HYBRID METAL OXIDE PARTICLES

(71) Applicant: BASF SE, Ludwigshafen am Rhein (DE)

(72) Inventors: Brigitte LINDEMANN, Grenzach-Wyhlen (DE); Bernd HERZOG, Grenzach-Wyhlen (DE); Rupa Hiremath DARJI, Tarrytown, NY (US); Michael David BURKE, Tarrytown, NY (US); Liangliang QU, Tarrytown, NY (US); Carmen A. HENDRICKS-GUY, Tarrytown, NY (US); Keith TASK, Beachwood, OH (US)

(21) Appl. No.: 18/729,532

(22) PCT Filed: Jan. 17, 2023
(86) PCT No.: PCT/EP2023/050925

§ 371 Date: Jul. 17, 2024

(30) Foreign Application Priority Data
Jan. 18, 2022 (EP) 22151999.4

(51) Int. Cl. A61K 8/02; A61K 8/25; A61K 8/29; A61Q 17/04

(52) U.S. Cl. CPC A61K 8/0283; A61K 8/25; A61K 8/29; A61Q 17/04; A61K 2800/412; A61K 2800/43; A61K 2800/651

(57) ABSTRACT

The present invention relates to a method for increasing sun protection factor (SPF) of a UV filter composition, use of hybrid metal oxide particles for increasing SPF of a UV filter composition and UV filter compositions comprising the hybrid metal oxide particles.

### FIELD OF THE INVENTION

The present invention relates to a method for increasing sun protection factor (SPF) of a UV filter composition, use of hybrid metal oxide particles for increasing SPF of a UV filter composition and UV filter compositions comprising the hybrid metal oxide particles.

### BACKGROUND OF THE INVENTION

UV filter compositions are used to prevent adverse effects of solar radiations on human skin. A wide variety of UV absorbers are available, and a UV filter composition often comprises one or more UV absorber/s to achieve a high UV protection, i.e., a high sun protection factor (SPF).

However, challenges still exist for obtaining a UV filter composition having a high SPF. Several UV filters are characterized by a low solubility in the commonly used formulation media, and hence incorporation of a high amount of such a low-solubility UV filter in a UV filter composition becomes difficult. Further, the highest amount of a UV filter that can be incorporated in a UV filter composition is controlled by government regulations. Therefore, there exists a need for methods for increasing the SPF of a UV filter composition.

An agent used for increasing SPF of a UV filter composition may lead to undesired effects such as scattering of solar radiations, thereby resulting in an unpleasant appearance such as whitening of face. Therefore, it is desired that the method for increasing the SPF of a UV filter composition maintains its transparency and does not lead to undesired effects such as whitening effect.

Accordingly, it is an object of the invention to provide a method for increasing SPF of a UV filter composition. Further, it is an object of the invention to provide a UV filter composition that is not associated with adverse appearance problems such as whitening effect.

### SUMMARY OF THE INVENTION

It has been surprisingly found that the sun protection factor (SPF) of a UV filter composition can be increased by adding hybrid metal oxide particles to the UV filter composition.

Accordingly, an aspect of the presently claimed invention is directed to a method for increasing the sun protection factor of a UV filter composition, the method comprising adding hybrid metal oxide particles to the UV filter composition, the hybrid metal oxide particles comprising a continuous matrix of at least one first metal oxide having embedded therein an array of metal oxide particles, the metal oxide particles comprising at least one second metal oxide; wherein the first metal oxide and the second metal oxide are independently selected from the group consisting of silica, titania, alumina, zirconia, ceria, iron oxides, zinc oxide, indium oxide, tin oxide, chromium oxide, and mixtures thereof.

Another aspect of the presently claimed invention is directed to use of hybrid metal oxide particles for increasing the sun protection factor of a UV filter composition, the hybrid metal oxide particles comprising a continuous matrix of at least one first metal oxide having embedded therein an array of metal oxide particles, the metal oxide particles comprising at least one second metal oxide; wherein the first metal oxide and the second metal oxide are independently at least one selected from the group consisting of silica, titania, alumina, zirconia, ceria, iron oxides, zinc oxide, indium oxide, tin oxide, chromium oxide, and mixtures thereof.

The hybrid metal oxide particles are non-porous.

Yet another aspect of the presently claimed invention is directed to a UV filter composition UV filter composition comprising the hybrid metal oxide particles in the range of 0.1 to 25.0 wt. % based on total weight of the UV filter composition, the hybrid metal oxide particles comprising a continuous matrix of at least one first metal oxide having embedded therein an array of metal oxide particles, the metal oxide particles comprising at least one second metal oxide; wherein the metal oxide is at least one independently selected from the group consisting of silica, titania, alumina, zirconia, ceria, iron oxides, zinc oxide, indium oxide, tin oxide, chromium oxide, and mixtures thereof.

### BRIEF DESCRIPTION OF DRAWINGS

FIG. 1 depicts relative absorption values (RAV) of the UV filter composition 1 (placebo), composition 2 (3 wt. % hybrid metal oxide particles according to example 1), and composition 3 (comparative).

FIG. 2 is a graph of the absorbance of the UV filter composition 1 (placebo), composition 2 (3 wt. % hybrid metal oxide particles according to example 1), and composition 3 (comparative) in the range from 290 to 450 nm.

FIG. 3 depicts a chart related to the lightness value L* of the UV filter compositions 1 (placebo), 2 (3 wt. % hybrid metal oxide particles according to example 1), and composition 3 (commercial UV protection boosts).

### DETAILED DESCRIPTION

Before the present compositions and formulations of the presently claimed invention are described, it is to be understood that this invention is not limited to the particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e., a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure, or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment but may refer to so.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

It has been found that hybrid metal oxide particles can be used to increase the SPF of a UV filter composition. The presently claimed invention provides a method for increasing SPF of a UV filter compositions and provides UV filter compositions having a high SPF. Further, it is observed that the resultant UV filter composition does not show adverse appearance effects such as the whitening effect, which is usually associated with the addition of agents that scatter radiations.

The hybrid metal oxide particles are structural colorants that interact with UV and visible radiations via light interference effects. Structural colorants are materials containing nano-scaled structured surfaces small enough to interfere with visible light and produce color. Bulk samples of hybrid metal oxide particles exhibit saturated color with reduced unwanted light scattering when porosity and/or microsphere diameter and/or pore diameter are within a certain range. As a result, the presence of hybrid metal oxide particles in a UV filter composition leads to a decrease in the amount of the radiations travelling through the UV filter composition layer. Thus, an overall decrease in transmittance of the dye or the UV absorber is achieved without increasing its concentration.

Accordingly, an aspect of the presently claimed invention is directed to a method for increasing the sun protection factor of a UV filter composition, the method comprising adding hybrid metal oxide particles to the UV filter composition, the hybrid metal oxide particles comprising a continuous matrix of at least one first metal oxide having embedded therein an array of metal oxide particles, the metal oxide particles comprising at least one second metal oxide; wherein the first metal oxide and the second metal oxide are independently selected from the group consisting of silica, titania, alumina, zirconia, ceria, iron oxides, zinc oxide, indium oxide, tin oxide, chromium oxide, and mixtures thereof.

The hybrid metal oxide particles are non-porous.

Another aspect of the presently claimed invention is directed to use of hybrid metal oxide particles for increasing the sun protection factor of a UV filter composition, the hybrid metal oxide particles comprising a continuous matrix of at least one first metal oxide having embedded therein an array of metal oxide particles, the metal oxide particles comprising at least one second metal oxide; wherein the first metal oxide and the second metal oxide are independently at least one selected from the group consisting of silica, titania, alumina, zirconia, ceria, iron oxides, zinc oxide, indium oxide, tin oxide, chromium oxide, and mixtures thereof.

The hybrid metal oxide particles are non-porous.

In the context of the present invention, the SPF factor (sun protection factor, SPF) serves to evaluate light protection preparations (UV filter compositions) on humans (in vivo). It indicates how much longer a person with a UV filter agent can be exposed to the sun without suffering sunburn than would be possible with the particular individual's self-protection time.

The SPF is determined in vitro by measuring the diffuse transmission in the spectral range between 290 and 400 nm.

In the context of the present invention, the term “monodisperse” in reference to spheres, microspheres or nanospheres means particles having generally uniform shapes and generally uniform diameters. A present monodisperse population of spheres, microspheres or nanospheres may have 90%, 91%, 92%, 93%, 94%, 95%, 96%, 97%, 98% or 99% of the particles by number having diameters within ±7%, ±6%, ±5%, ±4%, ±3%, ±2% or +1% of the average diameter of the population.

In the context of the present invention, the terms ‘particles’, ‘microspheres’, ‘microparticles’, ‘nanospheres’, ‘nanoparticles’, ‘droplets’ etc. also refer to, for example, a plurality thereof, a collection thereof, a population thereof, a sample thereof, or a bulk sample thereof.

In the context of the present invention, the term ‘bulk sample’ refers to a population of particles. For example, a bulk sample of particles is simply a bulk population of particles, for example, ≥0.1 mg, ≥0.2 mg, ≥0.3 mg, ≥0.4 mg, ≥0.5 mg, ≥0.7 mg, ≥1.0 mg, ≥2.5 mg, ≥5.0 mg, ≥10.0 mg, or ≥25.0 mg. A bulk sample of particles may be substantially free of other components.

In the context of the present invention, the phrase “exhibits color observable by the human eye” means color will be observed by an average person. This may be for any bulk sample distributed over any surface area, for example, a bulk sample distributed over a surface area of, for example, from any of 1 cm², 2 cm², 3 cm², 4 cm², 5 cm², or 6 cm² to any of 7 cm², 8 cm², 9 cm², 10 cm², 11 cm², 12 cm², 13 cm², 14 cm², or 15 cm². It may also mean observable by a CIE 1931 2° standard observer and/or by a CIE 1964 10° standard observer. The background for color observation may be any background, for example, a white background, black background, or a dark background anywhere between white and black.

In the context of the present invention, the terms ‘micro’ or ‘micro-scaled’, for example, when referring to particles, mean from 1 micrometer (m) to less than 1000 μm. The term ‘nano’ or ‘nano-scaled’, for example, when referring to particles, mean from 1 nanometer (nm) to less than 1000 nm.

In the context of the present invention, the terms “microspheres”, “nanospheres”, “droplets”, etc., referred to herein may mean for example a plurality thereof, a collection thereof, a population thereof, a sample thereof or a bulk sample thereof.

In the context of the present invention, the term “micro” or “micro-scaled” means from 0.5 μm to 999 μm. The term “nano” or “nano-scaled” means from 1 nm to 999 nm.

In the context of the present invention, the terms “spheres” and “particles” may be interchangeable.

In the context of the present invention, the term “continuous matrix” means that the at least one first metal oxide forms a continuous assembly of particles as a uniform phase. This uniform oxide phase can be crystalline or amorphous. The oxide phase of the particles of the first metal oxide is essentially free of the second metal oxide. The second metal oxide particles are embedded within the continuous assembly of particles of the at least one first metal oxide and form distinct sections within the continuous matrix that exhibit a different refractive index than the continuous matrix itself. This is different to doped metal oxide materials in which a doping metal is included within the (crystal) structure of the metal oxide to be doped and both metals are homogenously distributed within the doped metal oxide.

Unless otherwise indicated, all parts and percentages mentioned herein are by weight. Weight percent (wt. %), if not otherwise indicated, is based on an entire composition free of any volatiles, that is, based on dry solids content.

In a preferred embodiment, the amount of the hybrid metal oxide particles is in the range from 0.1 to 25.0 wt. % based on total weight of the UV filter composition.

In a more preferred embodiment, the amount of the hybrid metal oxide particles is in the range from 0.5 to 10.0 wt. % based on total weight of the UV filter composition. In an even more preferred embodiment, the amount of the hybrid metal oxide particles is in the range from 1.0 to 8.0 wt. %; and most preferably 1.0 to 5.0 wt. % based on total weight of the UV filter composition.

In a preferred embodiment, the hybrid metal oxide particles have an average diameter in the range of 0.5 μm to 100.0 μm; more preferably 1.0 μm to 75.0 μm; even more preferably 2.0 m to 50.0 μm; and most preferably 3.0 μm to 25.0 jm.

In a preferred embodiment, the amount of the metal oxide in the hybrid metal oxide particles is in the range of 60.0 to 100.0 wt. % based on total weight of the hybrid metal oxide particles.

In a more preferred embodiment, the amount of the metal oxide in the hybrid metal oxide particles is in the range of 75.0 to 99.9 wt. %; even more preferably 90.0 to 99.9 wt. %; and most preferably 95.0 to 99.9 wt. % based on total weight of the hybrid metal oxide particles.

In a preferred embodiment, the amount of the first metal oxide is in the range from 2 to 90 wt. % based on total weight of the hybrid metal oxide particles. In a more preferred embodiment, the amount of the first metal oxide is in the range from 5 to 80 wt. %; even more preferably 7 to 70 wt. %; and most preferably 10 to 50 wt. % based on total weight of the hybrid metal oxide particles.

In a preferred embodiment, the first metal oxide is selected from silica, titania, alumina, zirconia, ceria, iron oxides, zinc oxide, indium oxide, tin oxide, chromium oxide, and mixtures thereof.

In a preferred embodiment, the first metal oxide is titania.

In a preferred embodiment, the amount of the second metal oxide is in the range of 10 to 98 wt. % based on total weight of the hybrid metal oxide particles. In a more preferred embodiment, the amount of the second metal oxide is in the range from 5 to 80 wt. %; even more preferably 7 to 70 wt. %; and most preferably 10 to 50 wt. % based on total weight of the hybrid metal oxide particles.

In a preferred embodiment, the first metal oxide particles fuse with each other during the preparation, and result in a continuous first metal oxide matrix.

In a preferred embodiment, the second metal oxide is selected from silica, titania, alumina, zirconia, ceria, iron oxides, zinc oxide, indium oxide, tin oxide, chromium oxide, and mixtures thereof.

In a preferred embodiment, the second metal oxide is silica.

In a preferred embodiment, the metal oxide particles comprising at least one second metal oxide have an average diameter in the range of 50 nm to 999 nm. In a preferred embodiment, the metal oxide particles comprising at least one second metal oxide have an average diameter in the range of 75 nm to 600 nm; and most preferably from 100 to 500 nm.

In a particularly preferred embodiment of the invention, the second metal oxide particles have an average diameter that is at least 3 times larger than the average diameter of the first metal oxide, preferably the second metal oxide has an average diameter is at least 5 times larger, more preferred at least 8 times larger, even more preferred at least 10 times larger, particularly preferred at least 12 times and most preferred at least 15 times larger than the average diameter of the first metal oxide. The smaller first metal oxides form the continuous matrix into which larger particles of the second metal oxide are embedded. The formation of the continuous matrix of the first metal oxide and embedding of the second metal oxide into the matrix works particularly well with the preferred differences in the average diameter between first and second metal oxide.

In a preferred embodiment, the weight ratio of the first metal oxide to the second metal oxide is in the range of 1:50 to 10:1. In a more preferred embodiment, the weight ratio of the first metal oxide to the second metal oxide is in the range of 1:20 to 10:1; even more preferably 1:10 to 10:1; and most preferably 1:4 to 4:1.

In a preferred embodiment, the weight ratio of the first metal oxide to the second metal oxide is 2:3.

In a preferred embodiment, the first metal oxide particles and/or the second metal oxide particles comprise combinations of different types of particles. For example, the first metal oxide particles can be a mixture of two different metal oxides (i.e., discrete distributions of metal oxide particles), such as a mixture of alumina particles and silica particles with each species being characterized by the same or similar size distributions.

In some embodiments, the first metal oxide particles and/or the second metal oxide particles comprise more complex compositions and/or morphologies. For example, the first metal oxide particles may comprise particles such that each individual particle comprises two or more metal oxides (e.g., silica-titania particles). Such particles may comprise, for example, an amorphous mixture of two or more metal oxides or may have a core-shell configuration (e.g., titania-coated silica particles, polymer-coated silica, carbon black-coated silica, etc.).

In a preferred embodiment, the continuous matrix further comprises at least one binder.

In a preferred embodiment, the binder is selected from silica, sodium silicate, magnesium silicate, calcium silicate, aluminum silicate, aluminum oxide hydroxide, sodium oxide, calcium carbonate, calcium aluminate, bentonite, kaolinite, montmorillonite, and combinations thereof.

In a preferred embodiment, the second metal oxide particles have a core-shell structure.

In a preferred embodiment, the second metal oxide particles are spherical metal oxide particles.

In a preferred embodiment, the array of the second metal oxide particles is an ordered array.

In a preferred embodiment, the array of the second metal oxide particles is a disordered array.

The terms “ordered array” and “disordered array” of the second metal oxide particles refer to the structural arrangement of the second metal oxide particles defined by the continuous matrix of at least the first metal oxide. In case of an “ordered array”, the second metal oxide particles are arranged in the continuous matrix in a repeating pattern. According to a preferred embodiment, such an ordered array results in an angle-dependent colour. In case of an “disordered array”, the second metal oxide particles are randomly distributed within the continuous matrix. According to a preferred embodiment, such a disordered array results in an angle-independent colour.

The term “surface functionalization” in the present invention represents a change in the surface of a material by bringing physical or chemical characteristics different from the ones originally found on the surface of the material. In the present invention, the surface modification is preferably the formation of a covalent bond with a surface functionalization agent (e.g. silane coupling agent as surface functionalization agent).

In a preferred embodiment, the second metal oxide particles have a surface functionalization. An example of a surface functionalization is a silane coupling agent (e.g., silane-functionalized silica).

In a preferred embodiment, the surface functionalization is performed on the first metal oxide particles and/or the second metal oxide particles prior to self-assembly and densification.

In some embodiments, the surface functionalization is performed on the hybrid metal oxide particles after densification.

In a preferred embodiment, the hybrid metal oxide particles comprise a surface functionalization.

In a preferred embodiment, the surface functionalization is carried out using a silane compound.

In a preferred embodiment, hybrid metal oxide particles are prepared by a method comprising:
generating liquid droplets from a particle dispersion comprising first metal oxide particles and second metal oxide particles;
drying the liquid droplets to provide dried particles comprising a discrete matrix of the first metal oxide particles embedded with the second metal oxide particles; and
heating the dried particles to obtain the hybrid metal oxide particles comprising a continuous matrix formed from the first metal oxide particles embedded with an array of the second metal oxide particles.

In a preferred embodiment, the step of heating the particles comprises sintering or calcining the dried particles to form the continuous matrix by densifying the first metal oxide particles.

In a preferred embodiment, the liquid droplets further comprise a binder, and the step of heating the dried particles facilitates forming the continuous matrix from the binder and the first metal oxide particles.

In a preferred embodiment, the first metal oxide particles have an average diameter from 1 nm to 120 nm. According to a more preferred embodiment of the invention, the first metal oxide particles have an average diameter of from 2 nm to 60 nm, even more preferred from 3 nm to 20 nm.

In a preferred embodiment, the second metal oxide particles have an average diameter from 50 nm to 999 nm.

In a preferred embodiment, one or more of the first metal oxide particles or the second metal oxide particles comprise a core-shell structure.

In a preferred embodiment, the step of generating liquid droplets is performed using a microfluidic process.

In a preferred embodiment, the step of generating and drying the liquid droplets is performed using a spray drying process.

In a preferred embodiment, the step of generating the liquid droplets is performed using a vibrating nozzle.

In a preferred embodiment, the step of drying the droplets comprises evaporation, microwave irradiation, oven drying, drying under vacuum, drying in the presence of a desiccant, or a combination thereof.

In a preferred embodiment, the liquid dispersion is an aqueous dispersion, an oil dispersion, an organic solvent dispersion, or a combination thereof.

In a preferred embodiment, the particle size ratio of the first metal oxide particles to the second metal oxide particles is from 1:20 to 1:5.

In a preferred embodiment, the hybrid metal oxide particles are prepared by a method comprising:
generating liquid droplets from a particle dispersion comprising a sol-gel matrix of a precursor of a first metal oxide and particles comprising a second metal oxide; and
drying the liquid droplets and densifying the sol-gel matrix into a continuous matrix to produce the hybrid metal oxide particles.

In a preferred embodiment, the precursor is at least one selected from a metal alkoxide or a metal chloride.

In a preferred embodiment, the second metal oxide particles are spherical metal oxide particles.

In a preferred embodiment, the hybrid metal oxide particles have an average diameter in the range of 0.5 μm to 100.0 μm;
comprise at least one first metal oxide as a continuous matrix;
comprise second metal oxide particles having an average diameter in the range of 50 nm to 999 nm; and
the second metal oxide particles are monodisperse.

In a preferred embodiment, the hybrid metal oxide particles
have an average diameter in the range of 0.5 μm to 100.0 μm;
comprise at least one first metal oxide as a continuous matrix;
comprise second metal oxide particles having an average diameter in the range of 50 nm to 999 nm; and
have a bimodal distribution of monodisperse second metal oxide particles.

In a preferred embodiment, the hybrid metal oxide particles have an average diameter in the range of 0.5 μm to 100.0 μm;
comprise at least one first metal oxide as a continuous matrix;
comprise second metal oxide particles having an average diameter in the range of 50 nm to 999 nm; and
the second metal oxide particles are polydisperse.

In a preferred embodiment, the hybrid metal oxide particles exhibit color in the visible spectrum at a wavelength in the range of 380 nm to 800 nm.

In a preferred embodiment, the hybrid metal oxide particles exhibit effect at a wavelength range in the ultraviolet spectrum in the range of 100 nm to 400 nm.

In a preferred embodiment, the hybrid metal oxide particles exhibit effect at a wavelength range in the ultraviolet and visible spectra in the range of 200 nm to 500 nm.

In a preferred embodiment, the hybrid metal oxide particles exhibit angle-dependent color. ‘Angle-dependent’ color means that observed color has dependence on the angle of incident light on a sample or on the angle between the observer and the sample.

In a preferred embodiment, the hybrid metal oxide particles exhibit angle-independent color. ‘Angle-independent’ color means that observed color has substantially no dependence on the angle of incident light on a sample or on the angle between the observer and the sample.

Angle-dependent color may be achieved, for example, with the use of monodisperse metal oxide particles (e.g., second metal oxide particles in the present embodiments). Angle-dependent color may also be achieved when a step of drying the liquid droplets is performed slowly, allowing the particles to become ordered.

Angle-independent color may be achieved when a step of drying the liquid droplets is performed quickly, not allowing the particles to become ordered.

The following embodiments may be utilized to achieve angle-dependent color resulting from ordered template particles, with the template and matrix particles comprising different metal oxides (e.g., titania matrix particles and silica template particles). As a first example embodiment of angle-dependent color, monodisperse and spherical template particles are embedded in matrix particles, and the matrix particles are subsequently densified. As a second example embodiment of angle-dependent color, two or more species of template particles that are collectively monodisperse and spherical are embedded in matrix particles, and the matrix particles are subsequently densified. Angle-dependent color is achieved independently of the polydispersity and shapes of the matrix particles.

The following embodiments may be utilized to achieve angle-independent color resulting from disordered template particles, with the template and matrix particles comprising different metal oxides (e.g., titania matrix particles and silica template particles). As a first example embodiment of angle-independent color, polydisperse template particles are embedded in matrix (e.g., metal oxide) particles, and the matrix particles are subsequently densified.

As a second example embodiment of angle-independent color, two different sized spherical template particles (i.e., a bimodal distribution of monodisperse template particles) are embedded in matrix particles, and the matrix particles are subsequently densified. The matrix particles may be spherical or non-spherical.

As a third example embodiment of angle-independent color, two different sized and polydisperse spherical template particles are embedded in matrix particles, and the matrix particles are subsequently densified.

Angle-independent color is achieved independently of the polydispersity and shapes of the matrix particles.

Any of the embodiments exhibiting angle-dependent or angle-independent color may be modified to exhibit whiteness or effects (e.g., reflectance, absorbance) in the ultraviolet spectrum.

In a preferred embodiment, the hybrid metal oxide particles further comprise a light absorber.

In a preferred embodiment, the light absorber is present in the range of 0.1 to 40.0 wt. %, more preferably 0.5 to 25.0 wt. %; and most preferably 1.0 to 10.0 wt. %.

In a preferred embodiment, the light absorber comprises at least one ionic species.

In a preferred embodiment, the UV filter composition comprises an UV absorber selected from the group consisting of
(d₁) p-aminobenzoic acid derivatives;
(d₂) salicylic acid derivatives;
(d₃) benzophenone derivatives;
(d₄) dibenzoylmethane derivatives;
(d₅) diphenyl acrylates;
(d₆) 3-imidazol-4-yl-acrylic acid and its esters;
(d₇) benzofuran derivatives;
(d₈) polymeric UV absorbers;
(d₉) cinnamic acid derivatives;
(d₁) camphor derivatives;
(d₁₁) hydroxyphenyltriazine derivatives;
(d₁) benzotriazole derivatives;
(d₁) trianilino-s-triazine derivatives;
(d₁₄) 2-phenylbenzimidazole-5-sulfonic acid and salts thereof;
(d₁) methyl o-aminobenzoates;
(d₁₆) homosalates;
(d₁) tris-biphenyltriazine derivatives;
(d₁₈) TiO₂, ZnO and mica;
(d₁₉) benzylidenemalonates;
(d₂₀) merocyanine derivatives;
(d₂₁) phenylene bis diphenyltriazines;
(d₂₂) imidazoline derivatives;
(d₂₃) diarylbutadiene derivatives;
(d₂₄) amino hydroxybenzoyl hexyl benzoate derivatives; and
(d₂₅) bis-(diethylamino hydroxybenzoyl benzoyl)-piperazine derivatives.

Examples of p-aminobenzoic acid derivatives (d₁) which can be employed according to the presently claimed invention, are 4-aminobenzoic acid (PABA); ethyldihydroxypropyl-PABA of the fromula

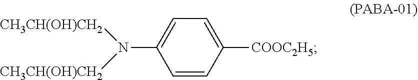

PEG-25-PABA of the formula

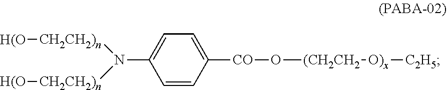

wherein m, n and x have the same meaning, and each denote an integer from 1 to 25; octyldimethyl PABA of the formula

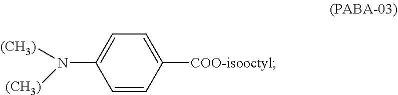

or glycyl aminobenzoate of the formula (PABA-04).

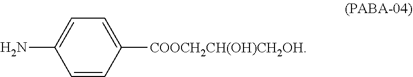

Example for salicylic acid derivatives (d₂) which can be employed according to the presently claimed invention, are
homomenthyl salicylate of the formula

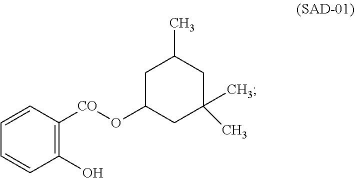 [SMILES: CC1CC(OC(=O)c2ccccc2O)CC(C)(C)C1]

triethanolamine salicylate of the formula (SAD-02)

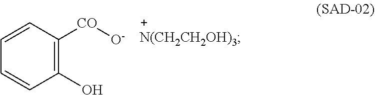

amyl p-dimethylaminobenzoate of the formula (SAD-03)

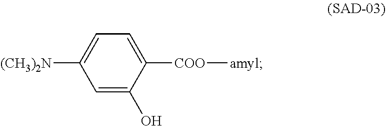

octyl salicylate of the formula

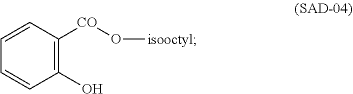

or 4-isopropylbenzyl salicylate of the formula

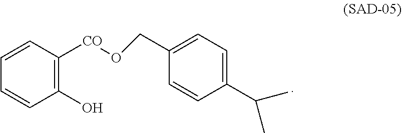 [SMILES: CC(C)c1ccc(COC(=O)c2ccccc2O)cc1]

Example for benzophenone derivatives (d₃) which can be employed according to the presently claimed invention, are:
benzophenone-3-(2-hydroxy-4-methoxybenzophenone); benzophenone-4-(2-hydroxy-4-methoxybenzophenone-5-sulfonic acid); benzophenone-8-(2,2′-dihydroxy-4-methoxybenzophenone); or amino-substituted hydroxybenzophenones of the formula

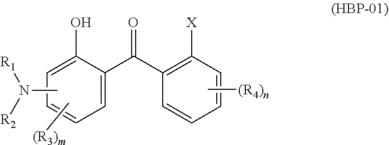

wherein
R₁ and R₂ denote hydrogen, C₁-C₂₀-alkyl, C₂-C₁₀-alkenyl, C₃-C₁₀-cycloalkyl, C₃-C₁₀-cycloalkenyl, wherein the substituents R₁ and R₂ together with the nitrogen atom to which they are bonded can form a 5- or 6-membered ring;
R₃ and R₄ independently of each other denote C₁-C₂₀-alkyl; C₂-C₁₀-alkenyl; C₃-C₁₀-cycloalkyl; C₃-C₁₀-cycloalkenyl; C₁-C₂₂-alkoxy; C₁-C₂₀-alkoxycarbonyl; C₁-C₁₂-alkylamino; C₁-C₁₂-dialkylamino; optionally substituted aryl; hetaryl; substituents conferring solubility in water selected from the group consisting of a nitrile group, and carboxylate, sulfonate or ammonium radicals;
X denotes hydrogen; COOR₅; or CONR₆R₇;
R₅, R₆, R₇ independently of each other denote hydrogen; C₁-C₂₀-alkyl; C₂-C₁₀-alkenyl; C₃-C₁₀-cycloalkyl; C₃-C₁₀-cycloalkenyl; (Y-O)ₒ-Z; or aryl;
Z denotes —CH₂—CH₃; —CH₂—CH₂—CH₃; —CH₂—CH₂—CH₂—CH₃; or —CH(CH₃)—CH₃;
m denotes 0 to 3;
n denotes 0 to 4; and
denotes 1 to 20.

In a more preferred embodiment, the UV absorber is 2-hydroxy-4-methoxybenzophenone-5-sulfonic acid.

Benzophenone derivatives (d₃) also include dimeric benzophenone derivatives corresponding to the formula

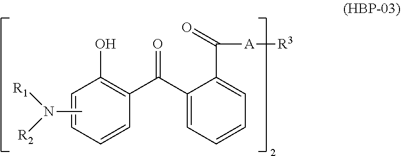

wherein
R₁ and R₂ independently of each other denote C₁-C₂₀-alkyl; C₂-C₂₀-alkenyl; C₃-C₁₀-cycloalkyl; C₃-C₁₀-cycloalkenyl; or R₁ and R₂ together with the nitrogen atom to which they are bonded form a 5- or 6-membered heterocyclic ring;
R₃ denotes alkylene, cycloalkylene, alkenylene or phenylene optionally substituted by a carbonyl or carboxyl group; a biradical of the formula (HBP-03a) *—CH₂—C≡C—CH₂—*; or

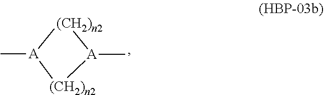

R₃ together with A forms a bivalent radical of the formula (HBP-03b)₂,
wherein
n₂ denotes an integer from 1 to 3;
A denotes —O—; or —N(R₅)—; and
R₅ denotes hydrogen; C₁-C₅-alkyl; or hydroxy-C₁-C₅-alkyl;
can also be employed according to the invention.

In a more preferred embodiment, dimeric benzophenone derivatives of the formula

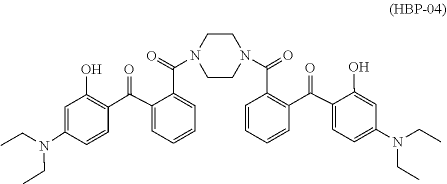 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

and

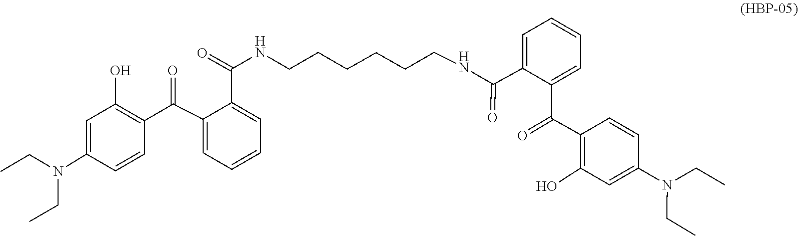 [SMILES: CCN(CC)c1ccc(C(=O)c2ccccc2C(=O)NCCCCCCNC(=O)c2ccccc2C(=O)c2ccc(N(CC)CC)cc2O)c(O)c1]

are employed as UV absorbers (d₃).

Examples of dibenzoylmethane derivatives (d₄) which can be employed according to the presently claimed invention are butylmethoxydibenzoylmethane-[1-(4-tert-butylphenyl)-3-(4-methoxyphenyl)propane-1,3-dione].

Examples of diphenylacrylate derivatives (d₅) which can be employed according to the presently claimed invention are octocrylene—(2-ethylhexyl 2-cyano-3,3′-diphenylacrylate) or etocrylene (ethyl 2-cyano-3,3′-diphenylacrylate).

Examples of benzofuran derivatives (d₇) which can be employed according to the presently claimed invention are 3-(benzofuranyl) 2-cyanoacrylate, 2-(2-benzofuranyl)-5-tert-butylbenzoxazole or 2-(p-aminophenyl)benzofuran and in particular the compounds of the formula

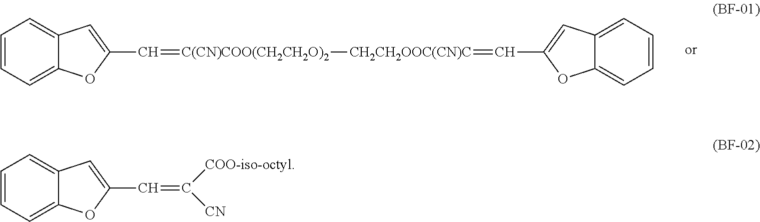

Examples of polymeric UV absorbers (d) which can be employed according to the presently claimed invention and contain one or more organosilicon radicals are benzylidenemalonate derivatives, in particular the compound of the formula

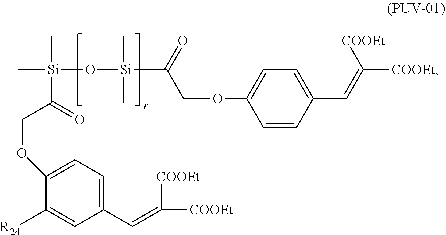

wherein R₂₄ denotes hydrogen or methoxy and r denotes approximately 7;
the compound of the formula

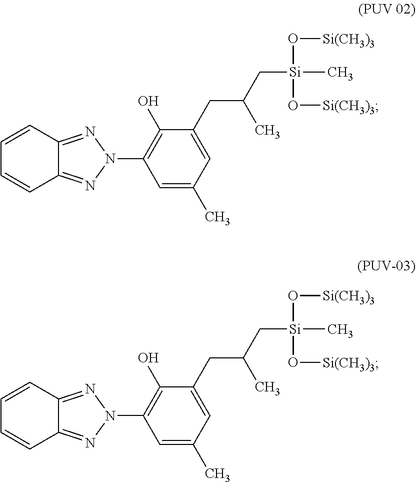

or
polysilicone-15 corresponding to the formula

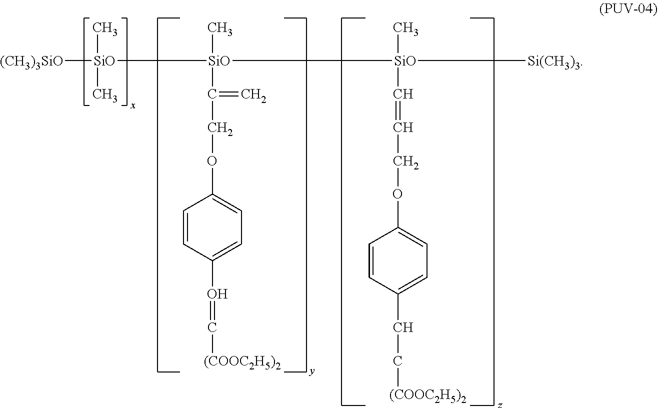

Examples of cinnamic acid esters (d₉) which can be employed according to the presently claimed invention are octyl methoxycinnamate (4-methoxycinnamic acid 2-ethylhexyl ester), diethanolamine methoxycinnamate (diethanolamine salt of 4-methoxycinnamic acid), isoamyl p-methoxycinnamate (4-ethoxycinnamic acid 2-isoamyl ester), 2,5-diisopropyl methycinnamate or a cinnamic acid amido derivative.

Examples of camphor derivatives (d₁₀) which can be employed according to the presently claimed invention are 4-methylbenzylidenecamphor-[3-(4′-methyl)benzylidenebornan-2-one], 3-benzylidenecamphor-(3-benzylidenebornan-2-one), polyacrylamidomethylbenzylidenecamphor {N-[2(and 4)-2-oxyborn-3-ylidenemethyl)benzyl]acrylamide polymer}, trimoniumbenzylidenecamphor sulfate—[3-(4′-trimethylammonium)-benzylidenebornan-2-one methylsulfate], terephthalydenedicamphorsulfonic acid {3,3′-(1,4-phenylenedimethine)-bis(7,7-dimethyl-2-oxobicyclo-[2.2.1]heptane-1-methanesulfonic acid} or salts thereof, or benzylidenecamphorsulfonic acid [3-(4′-sulfo)benzylidenebornan-2-one] or salts thereof.

Examples of hydroxyphenyltriazine derivatives (d₁₁) which can be employed according to the invention are, in particular, bis-resorcinyltriazines of the formula

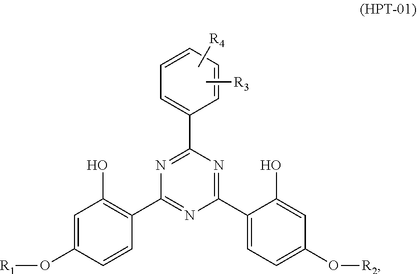

wherein
R₁ and R₂ independently of each other denote hydrogen; C₁-C₁₈-alkyl; C₂-C₁₈-alkenyl; a radical of the formula —CH₂—CH(—OH)—CH₂—O-T₁; a radical of the formula

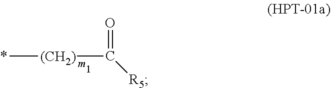

or a radical of the formula

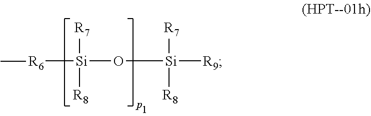

R₃, R₄ and R₅ independently of each other denote hydroxyl; C₁-C₅-alkoxy which is unsubstituted or substituted by one or more OH groups; amino; mono- or di-C₁-C₅-alkylamino; M; radical of the formula

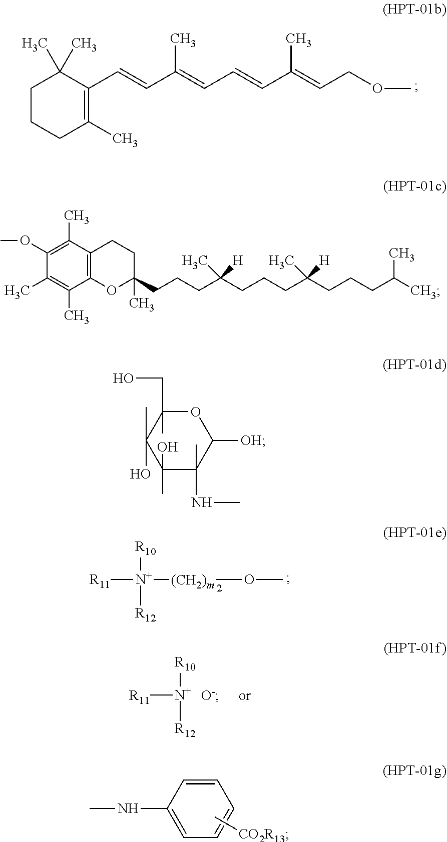

wherein R₁₀, R₁₁ and R₁₂ independently of each other denote C₁-C₁₄-alkyl which is unsubstituted or substituted by one or more OH groups;
R₁₃ denotes hydrogen; M; C₁-C₅-alkyl; or a radical of the formula —(CH₂)ₘ₃—O-T₁;
R₆ denotes the direct bond; a straight-chain or branched C₁-C₄-alkylene radical; or a radical of the formula —Cₘ₄H₂ₘ₄, or —Cₘ₄H₂ₘ₄—O—;
R₇, R₈ and R₉ independently of each other denote C₁-C₁₈-alkyl; C₁-C₁₈-alkoxy or a radical of the formula

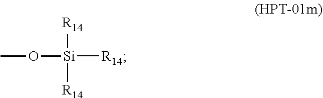

R₁₄ denotes C₁-C₅-alkyl;
M denotes a metal cation;
T₁ denotes hydrogen; or (C₁-C₈)-alkyl;
m₁, m₂ and m₃ independently of each other denote 1 to 3;
m₄ denotes 2 to 14; and
p₁ denotes 0 or a number from 1 to 5.

In a preferred embodiment, the compound class (d₁₁) are:
2-(4′-methoxyphenyl)-4,6-bis(2′-hydroxy-4′-n-octyloxyphenyl)-1,3,5-triazine;
2,4-bis{[4-(3-(2-propyIoxy)-2-hydroxypropyloxy)-2-hydroxy]phenyl}-6-(4-methoxyphenyl)-1,3,5-triazine;
2,4-bis{[4-(2-ethyIhexyIoxy)-2-hydroxy]phenyl}-6-[4-(2-methoxyethylcarboxyl)phenylamino]-1,3,5-triazine;
2,4-bis{[4-(tris(trimethylsiloxysilylpropyloxy)-2-hydroxy]phenyl}-6-(4-methoxyphenyl)-1,3,5-triazine;
2,4-bis{[4-(2″methylpropenyloxy)-2-hydroxy]phenyl}-6-(4-methoxyphenyl)-1,3,5-triazine;
2,4-bis{[4-(1′,1′,1′,3′,5′,5′,5′-heptamethytrisilyl-2″-methypropyoxy)-2-hydroxy]phenyl}-6-(4-methoxyphenyl)-1,3,5-triazine;
2,4-bis{[4-(3-(2-propyIoxy)-2-hydroxypropyloxy)-2-hydroxy]phenyl}-6-[4-ethylcarboxyl)phenylamino]-1,3,5-triazine;
2,4-bis{[4-(2-ethyIhexyIoxy)-2-hydroxy]phenyl}-6-(1-methylpyrrol-2-yI)-1,3,5-triazine; or
2,2′-[6-(4-methoxyphenyl)-1,3,5-triazine-2,4-diyl]bis[5-[(2-ethylhexyl)oxy]-(bis-ethylhexyloxyphenol methoxyphenyl triazine) corresponding to the formula

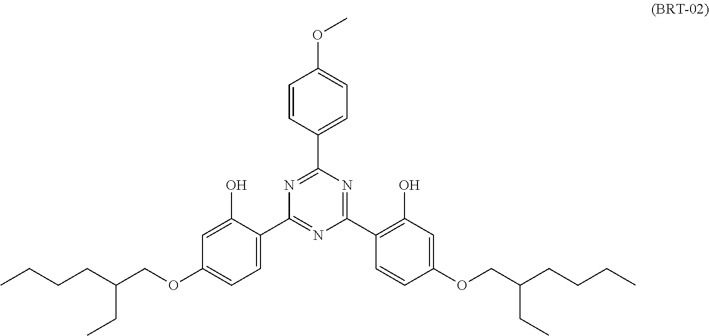 [SMILES: CCCCC(CC)COc1ccc(-c2nc(-c3ccc(OC)cc3)nc(-c3ccc(OCC(CC)CCCC)cc3O)n2)c(O)c1]

Examples of benzotriazole derivatives (d₁) which can be employed according to the presently claimed invention correspond to the formula

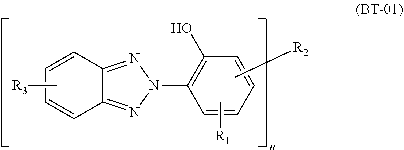

wherein
R₁ denotes hydrogen; C₁-C₁₂-alkyl; C₁-C₁₂-alkoxy; C₁-C₁₂-alkoxycarbonyl; C₅-C₁₀-cycloalkyl or —S0₃M;
R₃ denotes hydrogen; C₁-C₁₈-alkyl; C₁-C₁₂-alkoxy; or halogen; and
n denotes 1 or 2;
if n=1
R₂ denotes C₁-C₂₀-alkyl; C₅-C₁₀-cyclo-C₁-C₅-alkyl; C₁-C₁₂-alkoxy-C₁-C₅-alkyl; C₅-C₁₀-cycloalkoxy-C₁-C₅-alkyl; C₆-C₁₀-aryl; C₆-C₁₀-aryl-C₁-C₅-alkyl;
if n=2
R₂ denotes the direct bond; or —(CH₂)ₚ—; and
p is an integer from 1 to 3.

In a preferred embodiment, compounds of the formula (BT-01), wherein
R₁ denotes C₁-C₁₂-alkyl; or —S0₃M;
R₃ denotes hydrogen; halogen, preferably Cl;
n denotes 1;
R₂ C₁-C₁₂-alkyl; and
p denotes 1 to 3;
are possible.

In a more preferred embodiment, the benzotriazole derivatives (d₁₂) are compounds of the formula

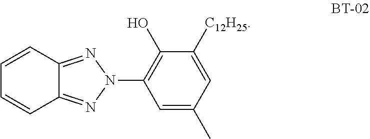

In a more preferred embodiment, UV filters of the formula BT-01 are compounds wherein
R₁ denotes hydrogen;
R₃ denotes C₁-C₁₈-alkyl;
n=2; and R₂ denotes —CH₂—.

In a more preferred embodiment, the benzotriazole derivatives (d₁₂) are compounds of the formula

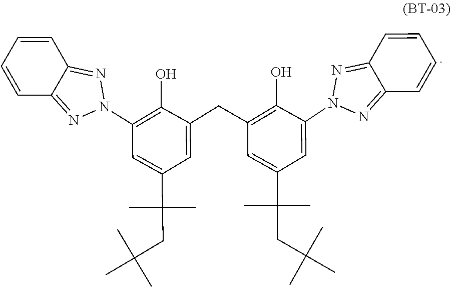 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

Examples of trianilino-s-triazine derivatives (d₁₃) which can be employed according to the presently claimed invention correspond to the formula

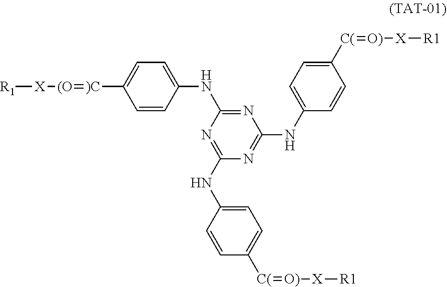

wherein
R₁, R₂ and R₃ independently of each other denote optionally substituted C₁-C₂₀-alkyl, aryl or hetaryl;
X denotes 0; or NR₄; and
R₄ denotes hydrogen; or optionally substituted C₁-C₂₀-alkyl, aryl or hetaryl.

In a preferred embodiment, trianilino-s-triazine derivatives (d₁) compound is ethylhexyl triazone corresponding to the formula

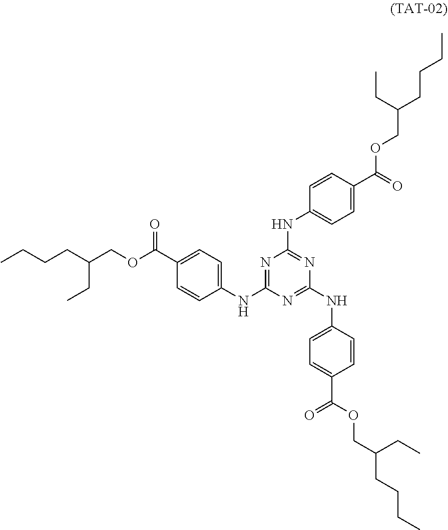 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

or Diethylhexyl butamido triazone corresponding to the formula

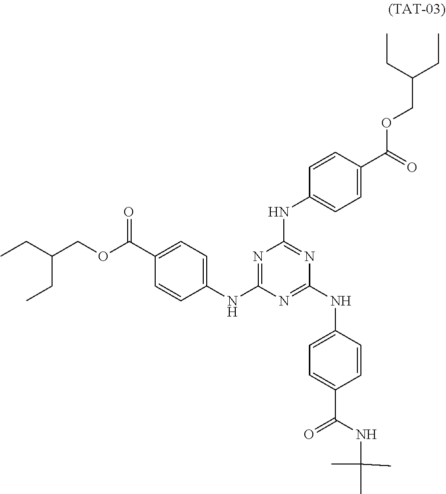 [SMILES: CCC(CC)COC(=O)c1ccc(Nc2nc(Nc3ccc(C(=O)NC(C)(C)C)cc3)nc(Nc3ccc(C(=O)OCC(CC)CC)cc3)n2)cc1]

or Ethylhexyl bis-lsopentylbenzoxazolylphenyl melamine corresponding to the formula (TAT-04)

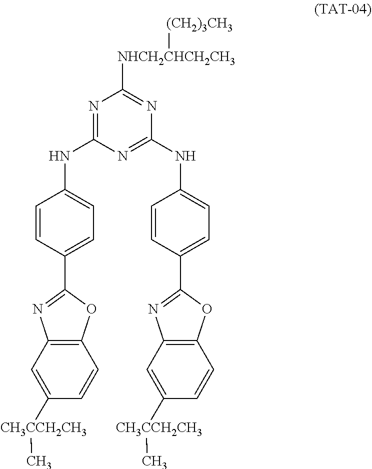

Examples of 2-phenylbenzimidazole-5-sulfonic acid, and salts thereof (d₁₄) which can be employed according to the invention is Disodium 2,2′-(1,4-phenylene)bis(6-sulfo-1H-1,3-benzimidazole-4-sulfonate (Bisdisulizole disodium).

Examples of tris-biphenyl-triazine derivatives (d₁₇) which can be employed according to the invention correspond to the formula

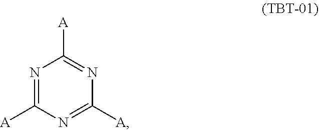

wherein

A denotes a radical of the formula

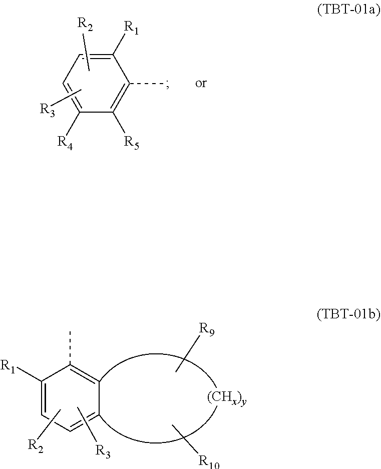

R₁ and R₅ independently of each other denote hydrogen; C₁-C₁₈-alkyl; or C₆-C₁₂-aryl;
R₂, R₃ and R₄ independently of each other denote hydrogen; or a radical of the formula

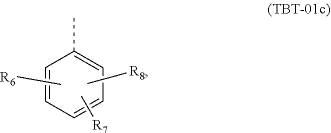

wherein in formula (TBT-01a) at least one of the radicals R₂, R₃ and R₄ denotes a radical of the formula (TBT-01c);
R₆, R₇, R₈, R₉ and R₁₀ independently of each other denote hydrogen; hydroxyl; halogen; C₁-C₁₈-alkyl; C₁-C₁₈-alkoxy; C₆-C₁₂-aryl; biphenylyl; C₆-C₁₂-aryloxy; C₁-C₁₈-alkylthio; carboxyl; -COOM; C₁-C₁₈-alkylcarboxyl; aminocarbonyl; or mono- or di-C₁-C₁₈-alkylamino; C₁-C₁₀-acylamino; —COOH;
M denotes an alkali metal ion;
x denotes 1 or 2; and
y denotes an integer from 2 to 10.

In a preferred embodiment, the UV filters (d₁₇) which can be employed according to the presently claimed invention correspond to the compounds of the formula

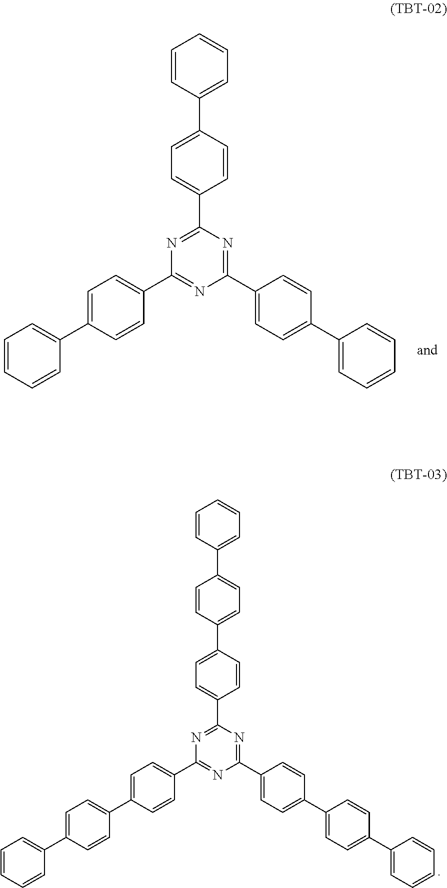 [SMILES: c1ccc(-c2ccc(-c3nc(-c4ccc(-c5ccccc5)cc4)nc(-c4ccc(-c5ccccc5)cc4)n3)cc2)cc1 ; c1ccc(-c2ccc(-c3ccc(-c4nc(-c5ccc(-c6ccc(-c7ccccc7)cc6)cc5)nc(-c5ccc(-c6ccc(-c7ccccc7)cc6)cc5)n4)cc3)cc2)cc1]

Examples of benzylidenemalonates (d₁₉) which can be employed according to the invention correspond to the formula

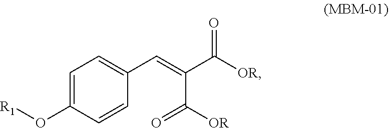

wherein
R₁ denotes methyl; ethyl; propyl; or n-butyl
if R₁ denotes methyl,
R denotes tert butyl;

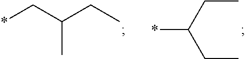

a radical of the formula

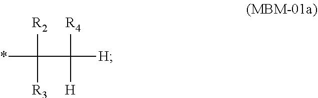

or a radical of the formula

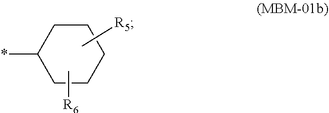

wherein
R₂ and R₃ independently of each other hydrogen; or methyl;
R₄ methyl; ethyl; or n-propyl;
R₅ and R₆ independently of each other hydrogen; or C₁-C₃-alkyl;
if R₁ denotes ethyl; propyl; or n-butyl,
R denotes isopropyl.

In a preferred embodiment, benzylidenemalonates (d₁₉) which can be employed according to the presently claimed invention are listed in the following table:

Examples of benzylidenemalonates which can be used
according to the presently claimed invention

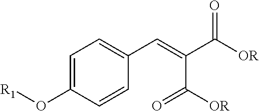

| | R₁ | R |
|---|---|---|
| (MBM-02) | methyl | 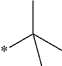 [SMILES: *C(C)(C)C] |
| (MBM-03) | methyl | 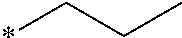 [SMILES: *CCC] |
| (MBM-04) | methyl | 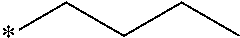 [SMILES: *CCCC] |
| (MBM-05) | methyl | 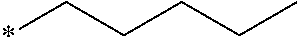 [SMILES: *CCCCC] |
| (MBM-06) | methyl | 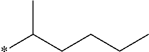 [SMILES: *C(C)CCCC] |
| (MBM-07) | methyl | 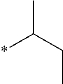 [SMILES: *C(C)CC] |
| (MBM-08) | methyl | 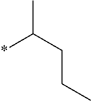 [SMILES: *C(C)CCC] |
| (MBM-09) | methyl | 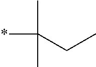 [SMILES: *C(C)(C)CC] |
| (MBM-10) | methyl | 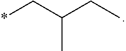 [SMILES: *CC(C)CC] |
| (MBM-11) | ethyl | 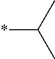 [SMILES: *C(C)C] |
| (MBM-12) | propyl | 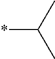 [SMILES: *C(C)C] |
| (MBM-13) | n-butyl | 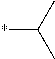 [SMILES: *C(C)C] |
| (MBM-14) | methyl | 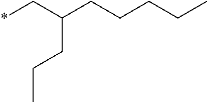 [SMILES: *CC(CCC)CCCCC] |
| (MBM-15) | methyl | 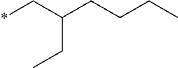 [SMILES: *CC(CC)CCCC] |

An example of the phenylene-bis-diphenyltriazines (d₂₁) which can be employed according to the presently claimed invention is 5,6,5,6-tetraphenyl-3,3′-(1,4-phenylene)-bis[1,2,4]triazine corresponds to the formula

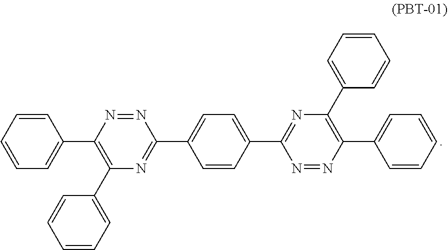 [SMILES: c1ccc(-c2nnc(-c3ccc(-c4nnc(-c5ccccc5)c(-c5ccccc5)n4)cc3)nc2-c2ccccc2)cc1]

An example of the imidazoline derivatives (d₂₂) which can be employed according to the presently claimed invention, is Ethylhexyldimethoxybenzylidenedioxoimidazoline propionate.

An example of the diarylbutadiene derivatives (d₂₃) which can be employed according to the presently claimed invention, is 1,1-dicarboxy-(2,2′-dimethylpropyl)-4,4-diphenylbutadiene.

Examples of amino hydroxybenzoyl hexyl benzoate derivatives (d₂₄) which can be employed according to the invention is 2-(4-Diethylamino-2-hydroxybenzoyl) benzoicacid hexylester corresponds to the formula.

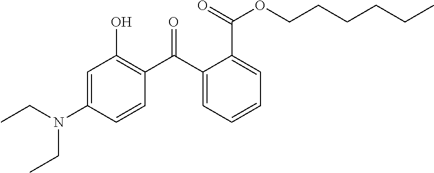 [SMILES: CCCCCCOC(=O)c1ccccc1C(=O)c1ccc(N(CC)CC)cc1O]

Examples of bis-(diethylamino hydroxybenzoyl benzoyl)-piperazine derivatives (d₂₅) which can be employed according to the invention corresponds to the formula

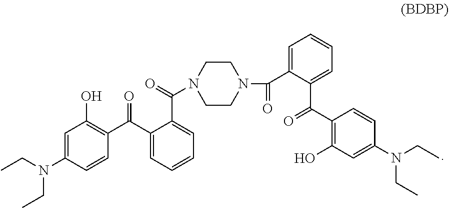 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

Each of the abovementioned UV filters (d₁)-(d₂₅) can be used according to the presently claimed invention as a mixture. For example, mixtures of two, three, four, five or six of the filter groups (d₁)-(d₂₅) can be used according to the presently claimed invention. Mixtures of two, three, four, five or six UV filters from one or more representatives of substance classes (d₁)-(d₂₅) can also be used according to the invention.

In a preferred embodiment, the UV filters (d) are representatives of the following compound classes:
(d₁) p-aminobenzoic acid derivatives;
(d₂) salicylic acid derivatives;
(d₃) benzophenone derivatives;
(d₄) dibenzoylmethane derivatives;
(d₅) diphenyl acrylates;
(d₆) 3-imidazol-4-yl-acrylic acid and its esters;
(d₇) benzofuran derivatives;
(d₉) cinnamic acid derivatives;
(d₁₀) camphor derivatives;
(d₁₁) hydroxyphenyltriazine derivatives;
(d₁₂) benzotriazole derivatives;
(d₁₃) trianilino-s-triazine derivatives;
(d₁₅) methyl o-aminobenzoates;
(d₁₆) homosalates;
(d₁₉) benzylidenemalonates; and
(d₂₀) merocyanine derivatives.

In a more preferred embodiment, the following oil-soluble UV filters are used according to the invention:
(d_{SOLL-1}) Benzophenone-3 (BP3);
(d_{SOL-2}) Benzophenone-4 (BP4);
(d_{SOL-3}) 3-Benzylidene Camphor (3BC);
(d_{SOL-4}) Bis-Ethylhexyloxyphenol Methoxyphenyl Triazine (BEMT);
(d_{SOL-5}) Butyl Methoxydibenzoylmethane (BMBM);
(d_{SOL-6}) Diethylhexyl Butamido Triazone (DBT);
(d_{SOL-7}) Drometrizole Trisiloxane (DTS);
(d_{SOL-8}) Ethylhexyl Triazone (EHT);
(d_{SOL-9}) Ethylhexyl Methoxycinnamate;
(d_{SOL-10}) Benzylidenemalonate (BM);
(d_{SOL-11}) Diethylamino Hydroxy Benzoyl Hexyl Benzoate (DHHB);
(d_{SOL-12}) Octocrylene;
(d_{SOL-13}) Polysilicone-15;
(d_{SOL-14}) Homosalate;
(d_{SOL-15}) Ethylhexyl salicylate; and
(d_{SOL-16}) Merocyanine.

In a more preferred embodiment, the following particulate UV filters are used according to the invention:
Methylene Bis-Benzotriazolyl Tetramethylbutylphenol (nano) (MBBT);
(TBT-02) Tris-Biphenyl Triazine (nano) (TBPT);
Bis-(Diethylaminohydroxybenzoyl Benzoyl) Piperazine (nano) (BDBP); and
Phenylene Bis-Diphenyltriazine (PBDT).

In a most preferred embodiment, the UV filter is at least one selected from the group consisting of
(d₉ₐ) Ethylhexyl Methoxycinnamate;
(d₁₁ₐ) Bis-Ethylhexyloxyphenol Methoxyphenyl Triazine;
(d₁₃ₐ) Ethylhexyl Triazone;
(d₃ₐ) Diethylamino Hydroxy Benzoyl Hexyl Benzoate;
Merocyanine;
Methylene Bis-Benzotriazolyl Tetramethylbutylphenol (nano) (MBBT);
(TBT-02) Tris-Biphenyl Triazine (nano) (TBPT); and
Bis-(Diethylaminohydroxybenzoyl Benzoyl) Piperazine (nano) (BDBP).

In a particularly preferred embodiment, the UV filter is a mixture of UV filters selected from the group consisting of (d₉ₐ), (d₁₁), (d₁₃ₐ) and (d₃ₐ).

In a preferred embodiment, the method or use is employed for protecting the skin against ultraviolet radiations and high energy visible light.

In a preferred embodiment, the method or use is employed for protecting the skin against ultraviolet radiations having a wavelength in the range from 280 and 400 nm, and high energy visible light in the range of wavelength from 380 to 480 nm.

The method or use is employed for protecting the skin against high energy visible light of wavelength in the range from 380 to 480 nm.

In a preferred embodiment, the method further minimizes or masks the whitening effect of the UV filter composition and maintains its transparency.

In a preferred embodiment, the use further minimizes or masks the whitening effect of the UV filter composition and maintains its transparency.

### UV Filter Compositions Comprising Hybrid Metal Oxide Particles

Another aspect of the presently claimed invention is directed to a UV filter composition comprising the hybrid metal oxide particles in the range of 0.1 to 25.0 wt. % based on total weight of the UV filter composition, the hybrid metal oxide particles comprising a continuous matrix of at least one first metal oxide having embedded therein an array of metal oxide particles, the metal oxide particles comprising at least one second metal oxide; wherein the first and the second metal oxide is at least one independently selected from the group consisting of silica, titania, alumina, zirconia, ceria, iron oxides, zinc oxide, indium oxide, tin oxide, chromium oxide, and mixtures thereof.

In a preferred embodiment, the hybrid metal oxide particles are non-porous.

In a more preferred embodiment, the amount of the hybrid metal oxide particles is in the range from 0.5 to 10.0 wt. % based on total weight of the UV filter composition. In an even more preferred embodiment, the amount of the hybrid metal oxide particles is in the range from 1.0 to 8.0 wt. %; and most preferably 1.0 to 5.0 wt. % based on total weight of the UV filter composition.

In a preferred embodiment, the UV filter composition comprises
a) water; and
b) the hybrid metal oxide particles in the range of 0.1 to 25.0 wt. % based on total weight of the UV filter composition.

In a preferred embodiment, the UV filter composition comprises
a) water; and
b) the hybrid metal oxide particles in the range of 0.5 to 10.0 wt. % based on total weight of the UV filter composition.

In a preferred embodiment, the UV filter composition comprises
a) water;
b) oil; and
c) the hybrid metal oxide particles in the range of 0.1 to 25.0 wt. % based on total weight of the UV filter composition.

In a preferred embodiment, oil is present in the form of a discontinuous phase in the range of 5.0 to 50.0 wt. %, based on total weight of the UV filter composition.

In a preferred embodiment, water is present in the form of a discontinuous phase in the range of 5.0 to 50.0 wt. %, based on total weight of the UV filter composition.

In a preferred embodiment, the UV filter composition comprises
a) oil; and
b) the hybrid metal oxide particles in the range of 0.1 to 25.0 wt. % based on total weight of the UV filter composition.

In a preferred embodiment, the UV filter composition comprises
a) oil; and
b) the hybrid metal oxide particles in the range of 0.5 to 10.0 wt. % based on total weight of the UV filter composition.

In a preferred embodiment, the hybrid metal oxide particles have an average diameter in the range of 0.5 μm to 100.0 μm.

In a preferred embodiment, the amount of the metal oxide in the hybrid metal oxide particles is in the range of 60.0 to 100.0 wt. %, based on total weight of the hybrid metal oxide particles.

In a preferred embodiment, the hybrid metal oxide particles have an average diameter in the range of 0.5 μm to 100.0 μm.

In a preferred embodiment, the hybrid metal oxide particles further comprise a light (UV-visible) absorber.

In a preferred embodiment, the light (UV-visible) absorber is present in the range of 0.1 wt. % to 40.0 wt. %.

In a preferred embodiment, the UV filter composition comprises an UV absorber selected from the group consisting of
(d₁) p-aminobenzoic acid derivatives;
(d₂) salicylic acid derivatives;
(d₃) benzophenone derivatives;
(d₄) dibenzoylmethane derivatives;
(d₅) diphenyl acrylates;
(d₆) 3-imidazol-4-yl-acrylic acid and its esters;
(d₇) benzofuran derivatives;
(d₈) polymeric UV absorbers;
(d₉) cinnamic acid derivatives;
(d₁₀) camphor derivatives;
(d₁₁) hydroxyphenyltriazine derivatives;
(d₁₂) benzotriazole derivatives;
(d₁₃) trianilino-s-triazine derivatives;
(d₁₄) 2-phenylbenzimidazole-5-sulfonic acid and salts thereof;
(d₁₅) methyl o-aminobenzoates;
(d₁₆) homosalates;
(d₁₇) tris-biphenyltriazine derivatives;
(d₁₈) TiO₂, ZnO and mica;
(d₁₉) benzylidenemalonates;
(d₂₀) merocyanine derivatives;
(d₂₁) phenylene bis diphenyltriazines;
(d₂₂) imidazoline derivatives;
(d₂₃) diarylbutadiene derivatives;
(d₂₄) amino hydroxybenzoyl hexyl benzoate derivatives; and
(d₂₅) bis-(diethylamino hydroxybenzoyl benzoyl)-piperazine derivatives.

Representative examples of the UV absorbers are described hereinabove.

In a preferred embodiment, the UV filter composition is a sunscreen composition.

In a preferred embodiment, the UV filter composition is day care composition.

In a preferred embodiment, the UV filter composition is at least one selected from the group consisting of creams, gels, lotions, alcoholic solutions, aqueous/alcoholic solutions, emulsions, wax/fat compositions, stick preparations, powders and ointments.

### Presentation Forms

The final UV filter composition listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellant gas or pump-action spray) or an aerosol,
in the form of a foam, or
in the form of a paste.

Of special importance as UV filter compositions for the skin are light-protective preparations, such as sun milks, lotions, creams, oils, sunblocks or tropicals, pre-tanning preparations or after-sun preparations, also skin-tanning preparations, for example self-tanning creams. Of particular interest are sun protection creams, sun protection lotions, sun protection milk and sun protection preparations in the form of a spray.

In addition, the UV filter compositions may contain further adjuvants as described below.

### 1) Oil Phase

In the context of the present invention, possible oily substances are, for example, Guerbet alcohols based on fatty alcohols having 6 to 18, preferably 8 to 10 carbon atoms (e.g. Eutanol® G), esters of linear C₆-C₂₂-fatty acids with linear or branched C₆-C₂₂-fatty alcohols and esters of branched C₆-C₁₃-carboxylic acids with linear or branched C₆-C₂₂-fatty alcohols, such as e.g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. In addition, esters of linear C₆-C₂₂-fatty acids with branched alcohols, in particular 2-ethylhexanol, esters of C₃-C₃₈-alkylhydroxycarboxylic acids with linear or branched C₆-C₂₂-fatty alcohols, in particular diethylhexyl malate, esters of linear and/or branched fatty acids with polyhydric alcohols (such as e.g. propylene glycol, dimer diol or trimer triol) and/or Guerbet alcohols, triglycerides based on C₆-C₁₀-fatty acids, liquid mono/di/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, plant oils, branched primary alcohols, substituted cyclohexanes, linear and branched C₆-C₂₂-fatty alcohol carbonates, such as e.g. Dicaprylyl Carbonate (Cetiol® OE), Guerbet carbonates based on fatty alcohols having 6 to 18 preferably 8 to 10 C atoms, esters of benzoic acid with linear and/or branched C₆-C₂₂-alcohols (e.g. Finsolv® TN), linear or branched, symmetric or unsymmetric dialkyl ethers having 6 to 22 carbon atoms per alkyl group, such as e.g. dicaprylyl ether (Cetiol® OE), ring-opening products of epoxidized fatty acid esters with polyols (Hydagen® HSP, Sovermol® 750, Sovermol® 1102), silicone oils (cyclomethicone, silicon methicone types and others) and/or aliphatic or naphthenic hydrocarbons, such as e.g. mineral oil, vaseline, petrolatum, squalane, squalene, isohexadecane or dialkylcyclohexanes are suitable in consideration.

In certain embodiments, the oily substances are medium-polarity oils, in particular esters of C₂-C₁₂-dicarboxylic acids with linear or branched alcohols having 1 to 22 carbon atoms and/or linear and branched C₆-C₂₂-fatty alcohol carbonates, adipic acid esters of linear or branched alcohols having 1 to 22 carbon atoms, very particularly of linear alcohols having 1 to 6 carbon atoms, are particularly suitable here.

Linear and branched fatty alcohol carbonates, in particular dicaprylyl carbonate, are particularly preferably used as oily substance.

In a more preferred embodiment, dibutyl adipate is used as oily substance.

In an even more preferred embodiment, the oil phase is selected from C₁₂-15 alkyl benzoate, dibutyl adipate, dicaprylyl carbonate, propylheptyl caprylate, caprylic/capric triglyceride, dicaprylyl ether, butylene glycol dicaprylate/dicaprate, coco-caprylate, octyldodecanol, dipropylheptyl carbonate, caprylyl-caprylate/caprate, cocoglycerides, ethylhexyl stearate, isohexadecane, isopropyl palmitate, and isopropyl myristate.

In another embodiment, the amount of oil phase is in the range of 20 to 35 wt. %, based on total weight of the UV filter composition.

### 2) Surfactant

In a preferred embodiment, the UV filter composition further comprises at least one emulsifier in the range of 1.0 to 20.0 wt. % based on total weight of the UV filter composition.

In a preferred embodiment, the emulsifier is selected from the group consisting of an anionic emulsifier, a cationic emulsifier, a nonionic emulsifier, and a polymeric emulsifier.

The anionic surfactants are characterized by one or more anionic group which confers solubility in water, such as e.g., a carboxylate, sulfate, sulfonate or phosphate group, and a lipophilic radical. In addition, the molecule can contain polyglycol ether, ester, ether and hydroxyl groups. Anionic surfactants which are tolerated by skin are known to the person skilled in the art in large numbers from relevant handbooks and are commercially obtainable.

Representative examples of the preferred anionic surfactants are, in each case in the form of their salts, ether-carboxylic acids, acylsarcosides having 8 to 24 C-atoms in the acyl group, acyltaurides having 8 to 24 C-atoms in the acyl group, acylisethionates having 8 to 24 C-atoms in the acyl group, sulfosuccinic acid mono- and dialkyl esters having 8 to 24 C-atoms in the alkyl group and sulfosuccinic acid monoalkyl polyoxyethyl esters having 8 to 24 C-atoms in the alkyl group and 1 to 6 oxyethyl groups, linear alkanesulfonates having 8 to 24 C-atoms, linear alpha-olefinsulfonates having 8 to 24 C-atoms, alpha-sulfo-fatty acid methyl esters of fatty acids having 8 to 30 C-atoms, alkyl sulfates, alkyl polyglycol ether sulfates, esters of tartaric acid and citric acid, alkyl and/or alkenyl ether phosphates, sulfated fatty acid alkylene glycol esters, monoglyceride sulfates and monoglyceride ether sulfates as well as condensation products of C₈-C₃₀-fatty alcohols with protein hydrolysates and/or amino acids and derivatives thereof, so-called protein fatty acid condensates, e.g. Lamepon®, Gluadin®, Hostapon® KCG or Amisoft®.

The salts of these surfactants are preferably selected from the sodium, potassium and ammonium and the mono-, di- and trialkanalammonium salts having 2 to 4 C-atoms in the alkanol group.

Particularly suitable anionic surfactants are liquid at room temperature, preferably from 18 to 25° C. A desirable feature in particular of these anionic surfactants is that they have a low water content of at most 10 wt. %, preferably 0.1 to 5 wt. %, based on the total weight of the anionic surfactant.

In a most preferred embodiment, the anionic surfactants are alk(en)yl polyglycol ether citrates and in particular mixtures of mono-, di- and triesters of citric acid and alkoxylated alcohols which correspond to the formula (I):

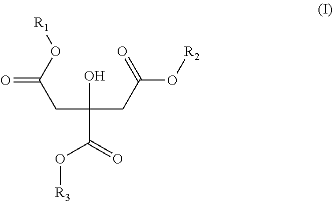

wherein
R₁, R₂ and R₃ independently of each other denote hydrogen or the radical of the formula (II) R₄(OCH₂CHR₅)ₙ
wherein
R₄ represents a linear or branched alkyl and/or alkenyl radical having 6 to 22 carbon atoms,
R₅ represents hydrogen or a methyl radical, and
n represents a number from 1 to 20, with the condition that at least one of the radicals
R₁, R₂ or R₃ is other than hydrogen.

Typical examples of the alcohol part of the esters are addition products of on average 1 to 20 mol, preferably 5 to 10 mol of ethylene oxide and/or propylene oxide on caproyl alcohol, capryl alcohol, 2-ethylhexyl alcohol, capric alcohol, lauryl alcohol, isotridecyl alcohol, myristyl alcohol, cetyl alcohol, palmitolelyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical grade mixtures thereof.

Such alk(en)yl polyglycol ether citrates are advantageous for the agents according to the invention since they are liquid anionic surfactants having a low water content of maximum 5 wt. %, based on the anionic surfactant.

The anionic surfactants are preferably present in amounts in the range of 7 to 17 wt. %, based on total weight of the UV filter composition.

The agents according to the invention furthermore comprise at least (c) 0.5 to 25 wt. % of a further co-surfactant which differs from anionic surfactants.

Suitable co-surfactants are, in principle, zwitterionic, ampholytic, cationic and/or nonionic surfactants.

Those surface active compounds which carry at least one quaternary ammonium group and at least one —COO⁽⁻⁾ or —SO₃⁽⁻⁾ group in the molecule are called zwitterionic surfactants. Particularly suitable zwitterionic surfactants are the so-called betaines, such as the N-alkyl-N,N-dimethylammonium glycinates, for example coco-alkyldimethylammonium glycinate, N-acylaminopropyl-N,N-dimethylammonium glycinates, for example coco-acylamimopropyldimethylammonium glycinate, and 2-alkyl-3-carboxymethyl-3-hydroxyethylimidazoline having in each case 8 to 18 C-atoms in the alkyl or acyl group, and coco-acylaminoethylhydroxyethylcarboxymethyl glycinate. The fatty acid amide derivative known under the INCI name Cocamidopropyl Betaine is a preferred zwitterionic surfactant. Tego® Betain 810 (INCI: Capryl/Capramidopropyl Betaine) and a surfactant mixture of Rewopol® SBCS 50K (INCI: Disodium PEG-5 Laurylcitrate Sulfosuccinate, Sodium Laureth Sulfate) and Tego® Betain 810 (Capryl/Capramidopropyl Betaine), in particular in the weight ratio of 1:4 to 4:1, very particularly preferably in the weight ratio of from 1:4 to 1:1, are particularly preferred according to the invention.

Ampholytic surfactants are understood as meaning those surface-active compounds which contain, apart from a C₈-C₁₈-alkyl or acyl group, at least one free amino group and at least one —COOH or —SO₃H group in the molecule and are capable of formation of inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case 8 to 18 C atoms in the alkyl group. Preferred ampholytic surfactants are N-coco-alkylaminopropionate, coco-acylaminoethylaminopropionate and C₁₂₋₁₈-acylsarcosine.

Quaternary ammonium compounds in particular can be used as cationic surfactants. Surfactants from this substance class have a particularly high affinity for the skin and can improve the degree of sensory smoothness. These include, inter alia, ammonium halides, in particular chlorides and bromides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. The very readily biodegradable quaternary ester compounds, such as, for example, the dialkylammonium methosulfates and methylhydroxyalkyldialkoyloxyalkylammonium methosulfates marketed under the trade name Stepantex® and the corresponding products of the Dehyquart® series, can furthermore be employed as cationic surfactants. The term “esterquats” is in general understood as meaning quaternized fatty acid triethanolamine ester salts. They impart to the compositions particularly soft feel. These are known substances which are prepared by the relevant methods of organic chemistry. Further cationic surfactants which can be used according to the invention are the quaternized protein hydrolysates.

Nonionic surfactants are particularly preferably present as co-surfactants, for example
addition products of from 2 to 50 mol of ethylene oxide and/or 0 to 20 mol of propylene oxide on linear fatty alcohols having 8 to 40 C atoms, on fatty acids having 12 to 40 C atoms and on alkylphenols having 8 to 15 C atoms in the alkyl group;
C_{12/18} fatty acid mono- and diesters of addition products of from 1 to 50 mol of ethylene oxide on glycerol; glycerol mono- and diesters and sorbitan mono- and diesters of saturated and unsaturated fatty acids having 6 to 22 carbon atoms and ethylene oxide addition products thereof,
alkyl mono- and oligoglycoside having 8 to 22 carbon atoms in the alkyl radical and ethoxylated analogues thereof; addition products of from 7 to 60 mol of ethylene oxide on castor oil and/or hydrogenated castor oil;
polyol and/or polyglycerol esters, such as e.g., polyglycerol diisostearate or polyglycerol dimerate or polyglycerol 12-hydroxystearate;
addition products of from 2 to 15 mol of ethylene oxide on castor oil and/or hydrogenated castor oil;
partial esters based on linear, branched, unsaturated or saturated C₆-C₂₂-fatty acids, ricinoleic acid and 12-hydroxystearic acid with, pentaerythritol, dipentaerythritol, sugar alcohols (e.g., sorbitol), alkyl glucosides (e.g., methyl glucoside, butyl glucoside, lauryl glucoside) and polyglucosides (e.g., cellulose), or mixed esters, such as e.g., glyceryl stearate citrate and glyceryl stearate lactate;
wool wax alcohols;
polysiloxane/polyalkyl polyether copolymers and corresponding derivatives;
mixed esters of pentaerythritol, fatty acids, citric acid and fatty alcohol and/or mixed esters of fatty acids having 6 to 22 carbon atoms, methylglucose and polyols, preferably glycerol or polyglycerol; and
polyalkylene glycols.

The addition products of ethylene oxide and/or of propylene oxide on fatty alcohols, fatty acids, alkylphenols, glycerol mono- and diesters and sorbitan mono- and diesters of fatty acids or on castor oil are known, commercially obtainable products. These are homologue mixtures, the average degree of alkoxylation of which corresponds to the ratio of the substance amounts of ethylene oxide and/or propylene oxide and substrate with which the addition reaction is carried out. They are W/O or O/W emulsifiers, depending on the degree of ethoxylation. For the preparations according to the invention, the reaction products with 1-100 mol of ethylene oxide are particularly suitable.

Advantageous compounds from the group of nonionic surfactants are partial esters of polyols, in particular of C₃-C₆-polyols, such as, for example, glyceryl monoesters, partial esters of pentaerythritol or sugar esters, e.g. sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical grade mixtures thereof. Addition products of from 1 to 30, preferably 5 to 10 mol of ethylene oxide on the sorbitan esters mentioned are also suitable nonionic surfactants.

Nonionic surfactants from the group of alkyl oligoglycosides are particularly skin-friendly and may therefore preferably be suitable in the context of the invention. C₈-C₂₂-alkyl mono- and oligoglycosides, their preparation and their use are known. Their preparation is carried out in particular by reaction of glucose or oligosaccharides with primary alcohols having 8 to 22 C atoms, preferably 12 to 22, and particularly preferably 12 to 18 C atoms. With respect to the glycoside radical, both monoglycosides in which a cyclic sugar residue is bonded glycosidically to the fatty alcohol and oligomeric glycosides having a degree of oligomerization of up to preferably 8 are suitable. The degree of oligomerization here is a statistical mean based on a conventional distribution of homologues for such technical grade products Products which are available under the name Plantacare® contain a glucosidically bonded C₈-C₁₆-alkyl group on an oligoglucoside radical, the average degree of oligomerization of which is 1 to 2. The acylglucamides derived from glucamine are also suitable as nonionic surfactants.

Nonionic surfactants, preferably polyol and/or polyglycerol esters, are very particularly preferably present as co-surfactants in the agents according to the invention as component (c), and/or alkyl oligoglycosides.

The polyol component of these surfactants can be derived from substances which have at least two, preferably 3 to 12 and in particular 3 to 8 hydroxyl groups and 2 to 12 carbon atoms. Typical examples are:
glycerol and polyglycerol;
alkylene glycols, such as, for example, ethylene glycol, diethylene glycol, propylene glycol;
methylol compounds, such as, in particular, trimethylolethane, trimethylolpropane, trimethylolbutane, pentaerythritol and dipentaerythritol;
alkyl oligoglucosides having 1 to 22, preferably 1 to 8 and in particular 1 to 4 carbons in the alkyl radical, such as, for example, methyl and butyl glucoside;
sugar alcohols having 5 to 12 carbon atoms, such as, for example, sorbitol or mannitol;
sugars having 5 to 12 carbon atoms, such as, for example, glucose or sucrose;
amino-sugars, such as, for example, glucamine.

Reaction products based on polyglycerol are of particular importance because of their excellent use properties.

The acid component of these surfactants can be derived from straight chain, branched, saturated and/or unsaturated carboxylic acids, optionally with functional groups, such as hydroxyl groups. The acid component is particularly preferably fatty acids having 12 to 22 carbon atoms, which optionally carry a hydroxyl group, and in particular hydroxystearic acid.

In a preferred embodiment of the invention the diester of polyhydroxystearic acid, polyglyceryl 2-dipolyhydroxystearate, which is marketed, for example, by BASF Personal Care and Nutrition GmbH under the name Dehymuls® PGPH, is used as a glyceryl ester.

In a preferred embodiment of the invention Eumulgin® SG, Eumulgin® Prisma, Emulgade® sucro and Emulgade® Sucro Plus are used as surfactants.

In the agents according to the invention the further co-surfactants are conventionally present in an amount in the range of 0.5 to 25 wt. %; more preferably in the range of 3.0 to 18 wt. %; and particularly preferably in the range of 7 to 18 wt. %.

### 3) Additives

In a preferred embodiment, the UV filter composition further comprises additives selected from the group consisting of thickener, active ingredients, preservatives, and perfumes.

### Thickeners

Suitable thickeners are anionic, zwitterionic, amphoteric and nonionic copolymers, such as, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, acrylamidopropyltrimethylammonium chloride/acrylate copolymers, octylacrylamide/methyl methacrylate/tert-butylaminoethyl methacrylate/2-hydroxypropyl methacrylate polymers, vinylpyrrolidone/vinyl acetate copolymers, vinylpyrrolidone/dimethylaminoethyl methacrylate/vinylcaprolactam terpolymers and optionally polysaccharides, in particular xanthan gum, guar and guar derivatives, agar-agar, alginates and tyloses, cellulose and cellulose derivatives, such as carboxymethylcellulose, carboxymethylcellulose and hydroxycellulose and moreover silicones.

Preferably, thickeners selected from the group of polyacrylates and crosslinked polyacrylates, such as Rheocare TTA®, Cosmedia© SP, Rheocare® C Plus, Tinovis® ADE, Tinovis® GTC, are added.

Thickeners from the group of polysaccharides, such as Keltrol® T or Rheocare® XG, and thickeners such as Hydagen® 558P, Hydagen® Clean, Rheocare® XGN, Tinovis® GTC, Cosmedia® ACE are furthermore preferred.

Preferably, the amounts of thickener are in the range from 0.5 to 5 wt. %, in particular from 1 to 4 wt. %, calculated as active substance and based on total weight of the UV filter composition.

The thickeners can be added to the concentrated agent before the dilution with water is carried out or can be contained in the water with which the dilution of the concentrated agent is carried out.

According to a preferred process variant, the concentrated agent is mixed with the thickener, and water for dilution is added to this mixture and the further formulation constituents are optionally stirred in.

According to another preferred process variant, the water, the thickener and optionally the other auxiliary substances are stirred with one another and the concentrated agent is added to this mixture.

The final UV filter formulations prepared by the process according to the invention are often particularly finely divided O/W emulsion having an average particle size of <10 μm, preferably <5 μm.

Active compounds Biogenic active compounds which are suitable according to the invention are to be understood as meaning, for example, tocopherol, tocopherol acetate, tocopherol palmitate, ascorbic acid, (deoxy)ribonucleic acid and fragmentation products thereof, P-glucans, retinol, bisabolol, allantoin, phytantriol, panthenol, AHA acids, amino acids, ceramides, pseudoceramides, essential oils, plant extracts, such as e.g., Prunus extract, Bambara nut extract and vitamin complexes. Such active compounds are employed in final UV filter formulations as agents which trap free radicals and serve to regenerate the skin.

### Preservatives

Suitable preservatives are, for example, phenoxyethanol, formaldehyde solution, parabens, pentanediol or sorbic acid and the silver complexes known by the name Surfacine®.

### Perfume Oils

Perfume oils which may be mentioned are natural, plant and animal as well as synthetic odoriferous substances or mixtures thereof. Natural odoriferous substances are obtained, inter alia, by extraction of flowers, stems, leaves, fruit, fruit peel, roots and resins of plants. Animal raw materials are furthermore possible, such as, for example, civet and castoreum. Typical synthetic odoriferous compounds are products of the ester, ether, aldehyde, ketone, alcohol and hydrocarbon type. Preferably, mixtures of various odoriferous substances which together generate a pleasant fragrance note are used.

In a preferred embodiment, the perfume is selected from limonene, citral, linalool, alphaisomethylionon, geraniol, citronellol, 2-isobutyl-4-hydroxy-4-methyltetrahydropyrane, 2-tert.pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl-C-butylphenylmethylpropionalcinnamal, amylsalicylate, amylcinnamylalcohol, anisealcohol, benzoin, benzylalcohol, benzylbenzoate, benzylcinnamate, benzylsalicylate, bergamot oil, bitter orange oil, butylphenylmethylpropiol, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylsalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, and vanillin.

### Auxiliary Substances

In certain embodiments, the final UV filter formulations further comprise auxiliary substances, such as moisture-retaining agents/skin-moisturizing agents, viscosity regulators, oils, fats and waxes, surfactants, pearlescent waxes, super-oiling agents, stabilizers, cationic, zwitterionic or amphoteric polymers, further UV filters, biogenic active compounds, film-forming agents, swelling agents, hydrotropic substances, preservatives, solubilizers, perfume oils, dyestuffs, insect repellant active compounds etc., which are listed below by way of example.

Moisture-retaining agents serve to further optimize the sensory properties of the composition and for moisture regulation of the skin. The moisture-retaining agents can be present in an amount in the range of 0 to 5.0 wt. %, based on total weight of the UV filter composition.

Suitable substances are, inter alia, amino acids, pyrrolidonecarboxylic acid, lactic acid and salts thereof, lactitol, urea and urea derivative, uric acid, glucosamine, creatinine, collagen cleavage products, chitosan or chitosan salts/derivatives, and in particular polyols and polyol derivatives (e.g. glycerol, diglycerol, triglycerol, ethylene glycol, propylene glycol, butylene glycol, erythritol, 1,2,6-hexanetriol, polyethylene glycols, such as PEG-4, PEG-6, PEG-7, PEG-8, PEG-9, PEG-10, PEG-12, PEG-14, PEG-16, PEG-18, PEG-20), sugars and sugar derivatives (inter alia fructose, glucose, maltose, maltitol, mannitol, inositol, sorbitol, sucrose, sorbitylsilanediol, sucrose, trehalose, xylose, xylitol, glucuronic acid and salts thereof), ethoxylated sorbitol (sorbeth-6, sorbeth-20, sorbeth-30, sorbeth-40), honey and hardened honey, hardened starch hydrolysates and mixtures of hardened wheat protein and PEG-20/acetate copolymer. Substances which are preferably suitable according to the invention as moisture-retaining agents are glycerol, diglycerol, triglycerol and butylene glycol.

Possible insect repellants are, for example, N,N-diethyl-m-toluamide, 1,2-pentanediol or 3-(N-n-butyl-N-acetylamino)propionic acid ethyl ester), which is marketed by Merck KGaA under the name Insect Repellent 3535, and butylacetylaminoproprionate. They are conventionally employed in the compositions according to the invention in an amount in the range of 0 to 6 wt. %, based on total weight of the UV filter composition.

The viscosity of the agents according to the invention can be achieved by addition of viscosity regulators. Possible viscosity regulators are, inter alia, agents which impart consistency, such as e.g., fatty alcohols or hydroxy-fatty alcohols having 12 to 22 and preferably 16 to 18 carbon atoms and partial glycerides, fatty acids having 12 to 22 carbon atoms or 12-hydroxy-fatty acids. A combination of these substances with alkyl oligoglucosides and/or fatty acid Nmethylglucamides of the same chain length is also suitable, since such combinations deliver particularly stable and homogeneous emulsions. The viscosity regulators also include thickening agents, such as, for example, Aerosil types (hydrophilic silicic acids), polysaccharides, in particular xanthan gum, guar-guar, agar-agar, alginates and tyloses, carboxymethylcellulose and hydroxyethyl- and hydroxypropylcellulose, furthermore higher molecular weight polyethylene glycol mono- and diesters of fatty acids, polyacrylates (e.g. Carbopols® and Pemulen types from Goodrich; Synthalens® from Sigma; Keltrol types from Kelco; Sepigel types from Seppic; Salcare types from Allied Colloids), non-crosslinked and polyol-crosslinked polyacrylic acids, polyacrylamides, polyvinyl alcohol and polyvinylpyrrolidone. Bentonites, such as e.g., Bentone® Gel VS-5PC (Rheox), which is a mixture of cyclopentasiloxane, Disteardimonium Hectorite and propylene carbonate, have also proved to be particularly effective. Surfactants, such as, for example, ethoxylated fatty acid glycerides, esters of fatty acids with polyols, such as, for example, pentaerythritol or trimethylolpropane, fatty alcohol ethoxylates with a narrowed homologue distribution, alkyl oligoglucosides and electrolytes, such as e.g., sodium chloride and ammonium chloride, can also be employed for regulation of the viscosity.

In the context of the invention fats and waxes are understood as meaning all lipids having a fat- or wax-like consistency which have a melting point above 20° C. These include, for example, the classic triacylglycerols, that is to say the triesters of fatty acids with glycerol, which can be of plant or animal origin. These can also be mixed esters, that is to say triesters of glycerol with various fatty acids, or a mixture of various glycerides. These also include mixtures of mono-, di- and triglycerides. So-called hardened fats and oils which are obtained by partial hydrogenation are particularly suitable according to the invention. Hardened fats and oils of plants are preferred, e.g., hydrogenated castor oil, groundnut oil, soya oil, rape oil, beet seed oil, cottonseed oil, soya oil, sunflower oil, palm oil, palm kernel oil, linseed oil, almond oil, maize oil, olive oil, sesame oil, cacao butter and coconut fat. Oxidation-stable plant glycerides which are available under the name Cegesoft® or Novata® are particularly suitable.

Possible waxes are, inter alia, natural waxes, such as e.g. candelilla wax, carnauba wax, Japan wax, esparto grass wax, cork wax, guaruma wax, rice germ oil wax, sugar cane wax, ouricury wax, montan wax, beeswax, shellac wax, spermaceti, lanolin (wool wax), uropygium fat, ceresin, ozocerite (earth wax), petrolatum, paraffin waxes, microwaxes; chemically modified waxes (hard waxes), such as e.g. montan ester waxes, Sasol waxes, hydrogenated jojoba waxes and synthetic waxes, such as e.g. polyalkylene waxes and polyethylene glycol waxes.

In addition to the fats, fat-like substances, such as lecithins and phospholipids, are also possible as additives. Lecithins are glycero-phospholipids which are formed from fatty acids, glycerol, phosphoric acid choline by esterification, and are often also called phosphatidylcholines (PC). Cephalins, which are also called phosphatidic acids and are derivatives of 1,2-diacyl-sn-glycerol-3-phosphoric acids, may be mentioned as an example of natural lecithins. In contrast, phospholipids are usually understood as meaning mono- and preferably diesters of phosphoric acid with glycerol (glycerol phosphates). Sphingosines and sphingolipids are also possible as fat-like substances.

Suitable pearlescent waxes are, for example, alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxysubstituted carboxylic acids with C₆-C₂₂-fatty alcohols, specifically long-chain esters of tartaric acid; fatty substances, such as, for example, fatty alcohols, fatty ketones, fatty aldehydes, fatty ethers and fatty carbonates, which have at least 24 carbon atoms in total—specifically Lauron®; distearyl ether; fatty acids, such as stearic acid, C₁₂-C₂₂-hydroxy-fatty acids, behenic acid, ring-opening products of C₁₂-C₂₂-olefin epoxides with C₁₂-C₂₂-fatty alcohols and/or polyols having 2 to 15 carbon atoms and 2 to 10 hydroxyl groups and mixtures thereof.

Super-oiling agents which can be used are substances such as, for example, lanolin and lecithin and polyethoxylated or acylated derivatives of lanolin and lecithin, polyol fatty acid esters, monoglycerides and fatty acid alkanolamides, the latter simultaneously serving as foam stabilizers.

So-called stabilizers which can be employed are metal salts of fatty acids, such as e.g., magnesium, aluminum and/or zinc stearate or ricinoleate.

Suitable cationic polymers which further optimize the sensory properties of the compositions according to the invention and impart to the skin a sensation of softness are, for example, cationic cellulose derivatives, such as e.g. a quaternized hydroxyethylcellulose which is obtainable from Amerchol under the name Polymer JR 400®, cationic starch, copolymers of diallylammonium salts and acrylamides, quaternized vinylpyrrolidone/vinylimidazole polymers, such as e.g. Luviquat® (BASF), condensation products of polyglycols and amines, quaternized collagen polypeptides, such as, for example, Lauryldimonium Hydroxypropyl Hydrolyzed Collagen (Lamequat®L/Grinau), quaternized wheat polypeptides, polyethylenimine, cationic silicone polymers, such as e.g. amodimethicone, copolymers of adipic acid and dimethylaminohydroxypropyldiethylenetriamine (Cartaretine®/Sandoz), copolymers of acrylic acid with dimethyldiallylammonium chloride (Merquat® 550/Chemviron, polyaminopolyamides and crosslinked water-soluble polymers thereof, cationic chitin derivatives, such as, for example, quaternized chitosan, condensation products, optionally distributed in microcrystalline form, of dihaloalkyls, such as e.g. dibromobutane with bisdialkylamines, such as e.g. bis-dimethylamino-1,3-propane, cationic guar gum, such as e.g. Jaguar® CBS, Jaguar® C-17, Jaguar® C-16 from Celanese, quaternized ammonium salt polymers, such as e.g. Mirapol® A-15, Mirapol® AD-1, Mirapol® AZ-1 from Miranol.

Starch derivative can furthermore be employed to improve the skin sensation, e.g., Dry Flo@PC (INCI: Aluminum Starch Octenylsuccinate).

Suitable silicone compounds have already been mentioned with the oily substances. In addition to dimethylpolysiloxanes, methylphenylpolysiloxanes and cyclic silicones, amino-, fatty acid-, alcohol-, polyether-, epoxy-, fluorine-, glycoside- and/or alkyl-modified silicone compounds, which can be either liquid or resinous at room temperature, are also suitable. Simethicones, which are mixtures of dimethicones having an average chain length of from 200 to 300 dimethylsiloxane units and silicon dioxide or hydrogenated silicates, are furthermore suitable.

So-called film-forming agents which lead to a further improvement in the sensory properties of the preparations according to the invention are, for example, chitosan, microcrystalline chitosan, quaternized chitosan, collagen, hyaluronic acid and salts thereof and similar compounds, and the polyvinylpyrrolidones, vinylpyrrolidone/vinyl acetate copolymers, polymers of the acrylic acid series and quaternized cellulose derivatives already mentioned under the viscosity regulators.

To improve the flow properties of the compositions according to the invention hydrotropic substances, such as, for example, ethanol, isopropyl alcohol, or polyols, can furthermore be employed. Polyols which are possible here have preferably 2 to 15 carbon atoms and at least two hydroxyl groups. The polyols can also contain further functional groups, in particular amino groups, or can be modified with nitrogen.

Dyestuffs which can be used are the substances which are suitable and approved for cosmetic purposes.

The presently claimed invention offers one or more of the following advantages:
1. The present invention provides a method for increasing sun protection factor of a UV filter composition using hybrid metal oxide particles.
2. The method of the present invention increases SPF of a sunscreen formulation while minimizing or masking its whitening effect and maintaining its transparency.
3. The hybrid metal oxide particles of the present invention can be used for increasing SPF of a UV filter composition.
4. The hybrid metal oxide particles of the present invention are useful for increasing SPF of a UV filter composition while minimizing or masking its whitening effect and maintaining its transparency.

In the following, there are provided a list of embodiments to further illustrate the present disclosure without intending to limit the disclosure to specific embodiments listed below.
1. A method for increasing the sun protection factor of a UV filter composition, the method comprising adding hybrid metal oxide particles to the UV filter composition,
   the hybrid metal oxide particles comprising a continuous matrix of at least one first metal oxide having embedded therein an array of metal oxide particles, the metal oxide particles comprising at least one second metal oxide;
   wherein the first metal oxide and the second metal oxide are independently selected from the group consisting of silica, titania, alumina, zirconia, ceria, iron oxides, zinc oxide, indium oxide, tin oxide, chromium oxide, and mixtures thereof.
2. Use of hybrid metal oxide particles for increasing the sun protection factor of a UV filter composition,
   the hybrid metal oxide particles comprising a continuous matrix of at least one first metal oxide having embedded therein an array of metal oxide particles, the metal oxide particles comprising at least one second metal oxide;
   wherein the first metal oxide and the second metal oxide are independently at least one selected from the group consisting of silica, titania, alumina, zirconia, ceria, iron oxides, zinc oxide, indium oxide, tin oxide, chromium oxide, and mixtures thereof.
3. The method or use according to embodiment 1 or 2, wherein the hybrid metal oxide particles are non-porous.
4. The method or use according to any of embodiments 1 to 3, wherein the amount of the hybrid metal oxide particles is in the range from 0.1 to 25.0 wt. % based on total weight of the UV filter composition.
5. The method or use according to any of embodiments 1 to 4, wherein the amount of the hybrid metal oxide particles is in the range from 0.5 to 10.0 wt. % based on total weight of the UV filter composition.
6. The method or use according to any of embodiments 1 to 5, wherein the hybrid metal oxide particles have an average diameter in the range of 0.5 μm to 100.0 μm.
7. The method or use according to any of embodiments 1 to 6, wherein the amount of the metal oxide in the hybrid metal oxide particles is in the range of 60.0 to 100.0 wt. %, based on total weight of the hybrid metal oxide particles.
8. The method or use according to any of embodiments 1 to 7, wherein the amount of the first metal oxide is in the range from 2 to 90 wt. % based on total weight of the hybrid metal oxide particles.
9. The method or use according to any of embodiments 1 to 8, wherein the first metal oxide is selected from silica, titania, alumina, zirconia, ceria, iron oxides, zinc oxide, indium oxide, tin oxide, chromium oxide, and mixtures thereof.
10. The method or use according to any of embodiments 1 to 9, wherein the first metal oxide is titania.
11. The method or use according to any of embodiments 1 to 10, wherein the amount of the second metal oxide is in the range of 10 to 98 wt. % based on total weight of the hybrid metal oxide particles.
12. The method or use according to any of embodiments 1 to 11, wherein the second metal oxide is selected from silica, titania, alumina, zirconia, ceria, iron oxides, zinc oxide, indium oxide, tin oxide, chromium oxide, and mixtures thereof.
13. The method or use according to any of embodiments 1 to 12, wherein the second metal oxide is silica.
14. The method or use according to any of embodiments 1 to 13, wherein the metal oxide particles comprising at least one second metal oxide have an average diameter in the range of 50 nm to 999 nm.
15. The method or use according to any of embodiments 1 to 14, wherein the weight ratio of the first metal oxide to the second metal oxide is in the range of 1:50 to 10:1.
16. The method or use according to any of embodiments 1 to 15, wherein the weight ratio of the first metal oxide to the second metal oxide is 2:3.
17. The method or use according to any of embodiments 1 to 16, wherein the continuous matrix further comprises at least one binder.
18. The method or use according to embodiment 17, wherein the binder is selected from silica, sodium silicate, magnesium silicate, calcium silicate, aluminum silicate, aluminum oxide hydroxide, sodium oxide, calcium carbonate, calcium aluminate, bentonite, kaolinite, montmorillonite, and combinations thereof.
19. The method or use according to any of embodiments 1 to 18, wherein the second metal oxide particles have a core-shell structure.
20. The method or use according to any of embodiments 1 to 19, wherein the second metal oxide particles are spherical metal oxide particles.
21. The method or use according to any of embodiments 1 to 20, wherein the array of the second metal oxide particles is an ordered array.
22. The method or use according to any of embodiments 1 to 21, wherein the array of the second metal oxide particles is a disordered array.
23. The method or use according to any of embodiments 1 to 22, wherein the second metal oxide particles have a surface functionalization.
24. The method or use according to any of embodiments 1 to 23, wherein the hybrid metal oxide particles comprise a surface functionalization.
25. The method or use according to embodiment 23 or 24, wherein the surface functionalization is carried out using a silane compound.
26. The method or use according to any of embodiments 1 to 25, wherein the hybrid metal oxide particles
   a. have an average diameter in the range of 0.5 μm to 100.0 μm;
   b. comprise at least one first metal oxide as a continuous matrix;
   c. comprise second metal oxide particles having an average diameter in the range of 50 nm to 999 nm; and
   d. the second metal oxide particles are monodisperse.
27. The method or use according to any of embodiments 1 to 25, wherein the hybrid metal oxide particles
   a. have an average diameter in the range of 0.5 μm to 100.0 μm;
   b. comprise at least one first metal oxide as a continuous matrix;
   c. comprise second metal oxide particles having an average diameter in the range of 50 nm to 999 nm; and
   d. have a bimodal distribution of monodisperse second metal oxide particles.
28. The method or use according to any of embodiments 1 to 25, wherein the hybrid metal oxide particles
   a. have an average diameter in the range of 0.5 μm to 100.0 μm;
   b. comprise at least one first metal oxide as a continuous matrix;
   c. comprise second metal oxide particles having an average diameter in the range of 50 nm to 999 nm; and
   d. the second metal oxide particles are polydisperse.
29. The method or use according to any of the embodiments 1 to 28, wherein the hybrid metal oxide particles exhibit color in the visible spectrum at a wavelength in the range of 380 nm to 800 nm.
30. The method or use according to any of the embodiments 1 to 29, wherein the hybrid metal oxide particles exhibit effect at a wavelength range in the ultraviolet spectrum in the range of 100 nm to 400 nm.
31. The method or use according to any of the embodiments 1 to 29, wherein the hybrid metal oxide particles exhibit effect at a wavelength range in the ultraviolet and visible spectra in the range of 200 nm to 500 nm.
32. The method or use according to any of embodiments 1 to 31, wherein the UV filter composition further comprises an UV absorber selected from the group consisting of
   (d₁) p-aminobenzoic acid derivatives;
   (d₂) salicylic acid derivatives;
   (d₃) benzophenone derivatives;
   (d₄) dibenzoylmethane derivatives;
   (d₅) diphenyl acrylates;
   (d₆) 3-imidazol-4-yl-acrylic acid and its esters;
   (d₇) benzofuran derivatives;
   (d₈) polymeric UV absorbers;
   (d₉) cinnamic acid derivatives;
   (d₁₀) camphor derivatives;
   (d₁₁) hydroxyphenyltriazine derivatives;
   (d₁₂) benzotriazole derivatives;
   (d₁₃) trianilino-s-triazine derivatives;
   (d₁₄) 2-phenylbenzimidazole-5-sulfonic acid and salts thereof;
   (d₁₅) methyl o-aminobenzoates;
   (d₁₆) homosalates;
   (d₁₇) tris-biphenyltriazine derivatives;
   (d₁₈) TiO₂, ZnO and mica;
   (d₁₉) benzylidenemalonates;
   (d₂₀) merocyanine derivatives;
   (d₂₁) phenylene bis diphenyltriazines;
   (d₂₂) imidazoline derivatives;
   (d₂₃) diarylbutadiene derivatives;
   (d₂₄) amino hydroxybenzoyl hexyl benzoate derivatives; and
   (d₂₅) bis-(diethylamino hydroxybenzoyl benzoyl)-piperazine derivatives.
33. The method or use according to any of the embodiments 1 to 32 for protecting the skin against ultraviolet radiations and high energy visible light.
34. The method or use according to embodiment 33 for protecting the skin against ultraviolet radiations having a wavelength in the range from 280 and 400 nm, and high energy visible light in the range of wavelength from 380 to 480 nm.
35. The method or use according to embodiment 33 or 34 for protecting the skin against high energy visible light of wavelength in the range from 380 to 480 nm.
36. The method according to any of embodiments 1 to 35, wherein the method further minimizes or masks the whitening effect of the UV filter composition and maintains its transparency.
37. The use according to any of embodiments 1 to 35, wherein the use further minimizes or masks the whitening effect of the UV filter composition and maintains its transparency.
38. A UV filter composition comprising the hybrid metal oxide particles in the range of 0.1 to 25.0 wt. % based on total weight of the UV filter composition,
   the hybrid metal oxide particles comprising a continuous matrix of at least one first metal oxide having embedded therein an array of metal oxide particles, the metal oxide particles comprising at least one second metal oxide;
   wherein the first and the second metal oxide is at least one independently selected from the group consisting of silica, titania, alumina, zirconia, ceria, iron oxides, zinc oxide, indium oxide, tin oxide, chromium oxide, and mixtures thereof.
39. The UV filter composition according to embodiment 38 wherein the hybrid metal oxide particles are non-porous.
40. The UV filter composition according to embodiment 38 or 39 comprising
   a. water; and
   b. the hybrid metal oxide particles in the range of 0.1 to 25.0 wt. % based on total weight of the UV filter composition.
41. The UV filter composition according to embodiment 40 comprising
   a. water; and
   b. the hybrid metal oxide particles in the range of 0.5 to 10.0 wt. % based on total weight of the UV filter composition.
42. The UV filter composition according to embodiment 38 or 39 comprising
   a. water;
   b. oil; and
   c. the hybrid metal oxide particles in the range of 0.1 to 25.0 wt. % based on total weight of the UV filter composition.
43. The UV filter composition according to embodiment 42, wherein oil is present in the form of a discontinuous phase in the range of 5.0 to 50.0 wt. %, based on total weight of the UV filter composition.
44. The UV filter composition according to embodiment 42, wherein water is present in the form of a discontinuous phase in the range of 5.0 to 50.0 wt. %, based on total weight of the UV filter composition.
45. The UV filter composition according to embodiment 38 or 39 comprising
   a. oil; and
   b. the hybrid metal oxide particles in the range of 0.1 to 25.0 wt. % based on total weight of the UV filter composition.
46. The UV filter composition according to embodiment 45 comprising
   a. oil; and
   b. the hybrid metal oxide particles in the range of 0.5 to 10.0 wt. % based on total weight of the UV filter composition.
47. The UV filter composition according to any of embodiments 38 to 46, wherein the hybrid metal oxide particles have an average diameter in the range of 0.5 μm to 100.0 μm.
48. The UV filter composition according to any of the embodiments 38 to 47, wherein the amount of the metal oxide in the hybrid metal oxide particles is in the range of 60.0 to 100.0 wt. % based on total weight of the hybrid metal oxide particles.
49. The UV filter composition according to any of embodiments 38 to 48, wherein the UV filter composition further comprises an UV absorber selected from the group consisting of
   (d₁) p-aminobenzoic acid derivatives;
   (d₂) salicylic acid derivatives;
   (d₃) benzophenone derivatives;
   (d₄) dibenzoylmethane derivatives;
   (d₅) diphenyl acrylates;
   (d₆) 3-imidazol-4-yl-acrylic acid and its esters;
   (d₇) benzofuran derivatives;
   (d₈) polymeric UV absorbers;
   (d₉) cinnamic acid derivatives;
   (d₁₀) camphor derivatives;
   (d₁₁) hydroxyphenyltriazine derivatives;
   (d₁₂) benzotriazole derivatives;
   (d₁₃) trianilino-s-triazine derivatives;
   (d₁₄) 2-phenylbenzimidazole-5-sulfonic acid and salts thereof;
   (d₁₅) methyl o-aminobenzoates;
   (d₁₆) homosalates;
   (d₁₇) tris-biphenyltriazine derivatives;
   (d₁₈) TiO₂, ZnO and mica;
   (d₁₉) benzylidenemalonates;
   (d₂₀) merocyanine derivatives;
   (d₂₁) phenylene bis diphenyltriazines;
   (d₂₂) imidazoline derivatives;
   (d₂₃) diarylbutadiene derivatives;
   (d₂₄) amino hydroxybenzoyl hexyl benzoate derivatives; and
   (d₂₅) bis-(diethylamino hydroxybenzoyl benzoyl)-piperazine derivatives.
50. The UV filter composition according to any of embodiments 38 to 49, further comprising at least one emulsifier in the range of 1.0 to 20.0 wt. % based on total weight of the UV filter composition.
51. The UV filter composition according to embodiment 50, wherein the emulsifier is selected from the group consisting of an anionic emulsifier, a cationic emulsifier, a nonionic emulsifier, and a polymeric emulsifier.
52. The UV filter composition according to any of embodiments 38 to 51, further comprises additives selected from the group consisting of thickener, active ingredients, preservatives, and perfumes.
53. The UV filter composition according to any of embodiments 38 to 52, wherein the UV filter composition is a sunscreen composition.
54. The UV filter composition according to any of embodiments 38 to 52, wherein the UV filter composition is day care composition.
55. The UV filter composition according to any of the embodiments 38 to 54, wherein the UV filter composition is at least one selected fromcreams, gels, lotions, alcoholic solutions, aqueous/alcoholic solutions, emulsions, wax/fat compositions, stick preparations, powders and ointments.

While the presently claimed invention has been described in terms of its specific embodiments, certain modifications and equivalents will be apparent to those skilled in the art and are intended to be included within the scope of the presently claimed invention.

### EXAMPLES

The presently claimed invention is illustrated in detail by non-restrictive working examples which follow. More particularly, the test methods specified hereinafter are part of the general disclosure of the application and are not restricted to the specific working examples.

### Materials

The following materials are used in the Examples:
Eumulgin® Prisma is Disodium Cetearyl Sulfosuccinate;
Lanette® O is Cetyl stearyl Alcohol;
Cutina® PES is Pentaerythrityl Distearate;
Cetiol® B is Dibutyl Adipate;
Cetiol® Sensoft is Propylheptyl Caprylate;
Uvinul® T150 is Ethylhexyl Triazone;
Tinosorb® S is Bis-Ethylhexyloxyphenol Methoxyphenyl Triazine;
Uvinul® A Plus is Diethylamino Hydroxybenzoyl Hexyl Benzoate;
Rheocare® XGN is Xanthan Gum; and
Cosmedia® SP is Sodium Polyacrylate,
are available from BASF.

Euxyl® PE 9010 is Phenoxyethanol and Ethylhexylglycerin and is available from Ashland.

Neo Heliopan® OS is Ethylhexyl Salicylate and is available from Symrise.

### Methods

Average diameter or particle size: Particle size, as used herein, is synonymous with particle diameter and is determined, for example, by scanning electron microscopy (SEM) or transmission electron microscopy (TEM). Average particle size is synonymous with D₅₀, meaning half of the population resides above this point, and the other half resides below this point. Particle size refers to primary particles. Particle size may be measured by laser light scattering techniques with dispersions or dry powders.

Average porosity and average pore diameter: Mercury porosimetry analysis can be used to characterize the porosity of the particles. Mercury porosimetry applies controlled pressure to a sample immersed in mercury. External pressure is applied for the mercury to penetrate into the voids/pores of the material. The amount of pressure required to intrude into the voids/pores is inversely proportional to the size of the voids/pores. A mercury porosimeter generates volume and pore size distributions from the pressure versus intrusion data generated by the instrument using the Washburn equation. Porosity, as reported herein for hybrid metal oxide particles, is calculated as a ratio of unoccupied space and total particle volume. For example, porous silica particles containing voids/pores with an average size of 165 nm have an average porosity of 0.8.

### Determination of the In Vitro SPF of Formulation Examples

The determination of the in vitro SPF is performed by measuring the diffuse transmission in the UV-range using a Labsphere Ultraviolet Transmittance Analyzer 2000S. In order to simulate the inhomogeneous surface structure of human skin, substrates with rough or porous surface are taken for such measurements. For this method Sandblasted 4-5 μm PMMA (PolyMethylMethacrylate) plates, from Helioscience (France), are used as substrate.

The sunburn protection factor (SPF) formalism was first introduced by Sayre in 1979 [1], by which an average of the inverse transmission (1/T) of the respective sunscreen in the spectral range between 290 and 400 nm is calculated, including weighting with the irradiance spectrum of a UV source, Sₛ(λ), and the erythemal action spectrum, Sₑᵣ(λ):

$${SPF}_{\text{?}} = \frac{{\sum }_{\text{?}}^{\text{?}} {s}_{\text{?}} ( λ ) \cdot {S}_{\text{?}} ( λ )}{{\sum }_{\text{?}}^{\text{?}} {s}_{\text{?}} ( λ ) \cdot {S}_{\text{?}} ( λ ) \cdot T ( λ )}$$

$$\text{?} \text{indicates text missing or illegible when filed}$$

### REFERENCES

[1]R. M. Sayre, P. P. Agin, G. J. LeVee, E. Marlowe. A comparison of in vivo and in vitro testing of sunscreening formulas, Photochem. Photobiol. 29 (1979) 559-566

Transparency/whitening method: Color measurements were performed with the prepared compositions applied on PMMA plates also used for in vitro SPF measurement. From the obtained L*a*b* parameters L* refers to the lightness of a sample. The difference of L* to a blank sample is expressed as delta L* and can be used to compare the transparency or whitening of the samples.

### Experiments

### Example 1: Preparation of Hybrid Silica/Zinc Oxide Particles

An aqueous suspension of 100 nm spherical silica nanoparticles, and zinc oxide nanoparticles was prepared, which contained 1.8 wt. % of the 100 nm silica nanoparticles and 1.2 wt. % of the zinc oxide nanoparticles based on a total weight of the aqueous suspension. The aqueous suspension was spray dried under an inert atmosphere (nitrogen) at a 100° C. inlet temperature, a 40 mm spray gas pressure, a 100% aspirator rate, and a 30% flow rate (about 10 mL/min) using a BOCHI lab-scale spray dryer.

The spray dried powder was removed from the spray dryer's collection chamber and spread onto a silicon wafer for sintering. The spray dried powder was then calcined in a muffle furnace with a batch sintering process to densify and stabilize the particles. The heating parameters were as follows: the particles were heated from room temperature to 500° C. over a period of 4 hours, held at 500° C. for 2 hours, and then cooled back to room temperature over a period of 4 hours.

### Example 2: Hybrid Titania/Silica Particles with Angle-Dependent/Ordered Structure

An aqueous suspension of 180 nm spherical silica nanoparticles and 5 nm titania nanoparticles was prepared, which contained 1.8 wt. % of the silica nanoparticles and 1.2 wt. % of the titania nanoparticles based on a total weight of the aqueous suspension. The aqueous suspension was spray dried under an inert atmosphere (nitrogen) at a 1000 C inlet temperature, a 40 mm spray gas pressure, a 100% aspirator rate, and a 30% flow rate (about 10 mL/min) using a BÜCHI lab-scale spray dryer.

The spray dried powder was removed from the spray dryer's collection chamber and spread onto a silicon wafer for sintering. The spray dried powder was then calcined in a muffle furnace with a batch sintering process to densify and stabilize the particles. The heating parameters were as follows: the particles were heated from room temperature to 550° C. over a period of 12 hours, held at 550° C. for 2 hours, and then cooled back to room temperature over a period of 3 hours.

### Example 3: Disordered Hybrid Silica/Titania Particles

An aqueous suspension of 180 nm spherical silica nanoparticles, 160 nm spherical silica nanoparticles, and 5 nm titania nanoparticles was prepared, which contained 1.2 wt. % of the 180 nm silica nanoparticles, 0.6 wt. % of the 160 nm silica nanoparticles, and 1.2 wt. % of the titania nanoparticles based on a total weight of the aqueous suspension. The aqueous suspension was spray dried under an inert atmosphere (nitrogen) at a 1000 C inlet temperature, a 40 mm spray gas pressure, a 100% aspirator rate, and a 30% flow rate (about 10 mL/min) using a BÜCHI lab-scale spray dryer.

The spray dried powder was removed from the spray dryer's collection chamber and spread onto a silicon wafer for sintering. The spray dried powder was then calcined in a muffle furnace with a batch sintering process to densify and stabilize the particles. The heating parameters were as follows: the particles were heated from room temperature to 5500 C over a period of 7 hours, held at 5500 C for 2 hours, and then cooled back to room temperature over a period of 4 hours.

An SEM image of hybrid metal oxide particles demonstrated the presence of disordered template (silica) nanoparticles. The disordered particles display an angle independent blue coloration when dispersed in mineral oil with 1 wt. % carbon black per mass of colorant.

### Example 4: Hybrid Zinc Oxide/Silica Particles Produced Via a Sol-Gel Process

An aqueous suspension of 135 nm zinc oxide nanoparticles was prepared, which contained 1.8 wt. % of the 135 nm zinc oxide nanoparticles based on a total weight of the aqueous suspension. TEOS was then dissolved in the suspension at a concentration of 17.4 mg/mL. The aqueous suspension was spray dried under an inert atmosphere (nitrogen) at a 1000 C inlet temperature, a 40 mm spray gas pressure, a 100% aspirator rate, and a 30% flow rate (about 10 mL/min) using a BOCHI lab-scale spray dryer.

The spray dried powder was removed from the spray dryer's collection chamber and spread onto a silicon wafer for sintering. The spray dried powder was then calcined in a muffle furnace with a batch sintering process to densify and stabilize the particles. The heating parameters were as follows: the particles were heated from room temperature to 5000 C over a period of 4 hours, held at 500° C. for 2 hours, and then cooled back to room temperature over a period of 4 hours.

### Example 5: Hybrid Alumina/Silica Particles

An aqueous suspension of 300 nm spherical alumina nanoparticles and 5 nm silica nanoparticles was prepared, which contained 1.8 wt. % of the 300 nm alumina nanoparticles and 1.2 wt. % of the 5 nm silica nanoparticles based on a total weight of the aqueous suspension. The aqueous suspension was spray dried under an inert atmosphere (nitrogen) at a 1000 C inlet temperature, a 40 mm spray gas pressure, a 100% aspirator rate, and a 30% flow rate (about 10 mL/min) using a BOCHI lab-scale spray dryer.

The spray dried powder was removed from the spray dryer's collection chamber and spread onto a silicon wafer for sintering. The spray dried powder was then calcined in a muffle furnace with a batch sintering process to densify and stabilize the particles. The heating parameters were as follows: the particles were heated from room temperature to 5000 C over a period of 4 hours, held at 5000 C for 2 hours, and then cooled back to room temperature over a period of 4 hours.

### Determination of Properties

### Experiment 1: Light Attenuation by Hybrid Metal Oxide Particles

To determine the light attenuation properties, a dispersion (Dispersion 1) of the hybrid metal oxide particles obtained in Example 1 was prepared in an oil having a refractive index of ˜1.5 (such as mineral oil) at a concentration of at 1.67 mg/mL. For comparison, two dispersions (Dispersions 2 and 3) were prepared in the same manner as Dispersion 1 but using silica nanoparticles and zinc oxide nanoparticles respectively, instead of the hybrid metal oxide particles.

Dispersions 1, 2, and 3 were placed in separate wells into a UV-clear 96-well microtiter plate at 300 μL per well, and UV-visible light transmission through the well was measured via a plate reader spectrophotometer. The output is expressed as relative absorption value (RAV) units with subtraction of the blank, i.e., the UV-Vis light attenuation of the oil alone as background.

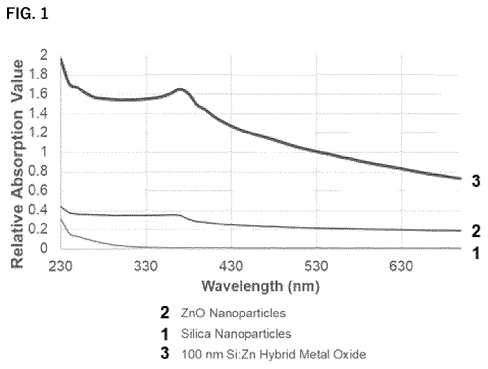

The RAV values of hybrid metal oxide particles, silica and zinc oxide particles are shown in FIG. 1. A higher RAV value indicated a greater attenuation of UV-light radiations.

It is clear from FIG. 1 that the hybrid metal oxide particles have a significantly higher RAV value as compared to the single metal oxide particles (silica and zinc oxide). Further, the RAV value of the hybrid metal oxide particles is higher than the addition of individual RAV values for the single metal oxide particles.

Experiment 2: Sun Protection Factor (SPF) Experiments with Hybrid SiO₂/ZnO Particles

The UV-filter compositions according to Table 1 were prepared for the evaluation of SPF of the hybrid metal oxide particles.

Composition 1 was basic (placebo) composition which contained the following UV filters: ethylhexyl triazone, bis-ethylhexyloxyphenol methoxyphenyl triazine, diethylamino hydroxybenzoyl hexyl benzoate and ethylhexyl salicylate. Suitable additives were also present in the composition.

Composition 2 was the composition of the presently claimed invention having 3.00 wt. % of hybrid metal oxide particles prepared according to Example 1.

Composition 3 is a composition for comparative analysis having 1.20 wt. % of commercial zinc oxide particles instead of the hybrid metal oxide particles. 1.20 wt. % Zinc oxide particles are equivalent to 3.0 wt. % of hybrid metal oxide particles of experiment 1.

| | | | | Composition 3 |
|---|---|---|---|---|
| | | Composition 1 | | Reference for |
| | | Placebo without | | comparative |
| Trade name | INCI | particles | Composition 2 | analysis |
| Eumulgin ® Prisma | Disodium Cetearyl | 1.00 | 1.00 | 1.00 |
| | Sulfosuccinate | | | |
| Lanette ® O | Cetearyl Alcohol | 2.00 | 2.00 | 2.00 |
| Cutina ® PES | Pentaerythrityl Distearate | 2.00 | 2.00 | 2.00 |
| Cetiol ® B | Dibutyl Adipate | 10.00 | 10.00 | 10.00 |
| Cetiol ® Sensoft | Propylheptyl Caprylate | 5.00 | 5.00 | 5.00 |
| Euxyl ® PE 9010 | Phenoxyethanol and | 1.00 | 1.00 | 1.00 |
| | Ethylhexylglycerin | | | |
| Uvinul ® T150 | Ethylhexyl Triazone | 3.00 | 3.00 | 3.00 |
| Tinosorb ® S | Bis-Ethylhexyloxyphenol | 3.00 | 3.00 | 3.00 |
| | Methoxyphenyl Triazine | | | |
| Uvinul ® A Plus | Diethylamino | 7.00 | 7.00 | 7.00 |
| | Hydroxybenzoyl Hexyl | | | |
| | Benzoate | | | |
| Neo Heliopan ® OS | Ethylhexyl Salicylate | 5.00 | 5.00 | 5.00 |
| Water | Aqua | 57.30 | 54.30 | 56.10 |
| Glycerine | Glycerine | 3.00 | 3.00 | 3.00 |
| Rheocare ® XGN | Xanthan Gum | 0.20 | 0.20 | 0.20 |
| Cosmedia ® SP | Sodium Polyacrylate | 0.50 | 0.50 | 0.50 |
| Particle | Hybrid metal oxide | — | 3.00 (=1.20 | — |
| | particles according to | | ZnO) | |
| | example 1 | | | |
| Z-Cote ® HP1 | ZnO | — | — | 1.20 |

The SPF values of these compositions was measured according to in vitro SPF method IS024444 and the results are summarized in Table 2.

TABLE 2
In vitro SPF evaluation of UV filter compositions

| | Composition 1 | | Composition 3 |
|---|---|---|---|
| | Placebo | | Reference for |
| | without | | comparative |
| Composition | particles | Composition 2 | analysis |
| Added | No particles | 3 wt.-% Hybrid metal | 1.2 wt.-% ZnO |
| particles | | oxide particles | particles |
| | | according to example 1 | |
| in vitro SPF | 32.2 | 45.5 | 41.0 |
| (ISO 24444) | | | |
| Delta L* | 7.7 | 8.3 | 8.1 |

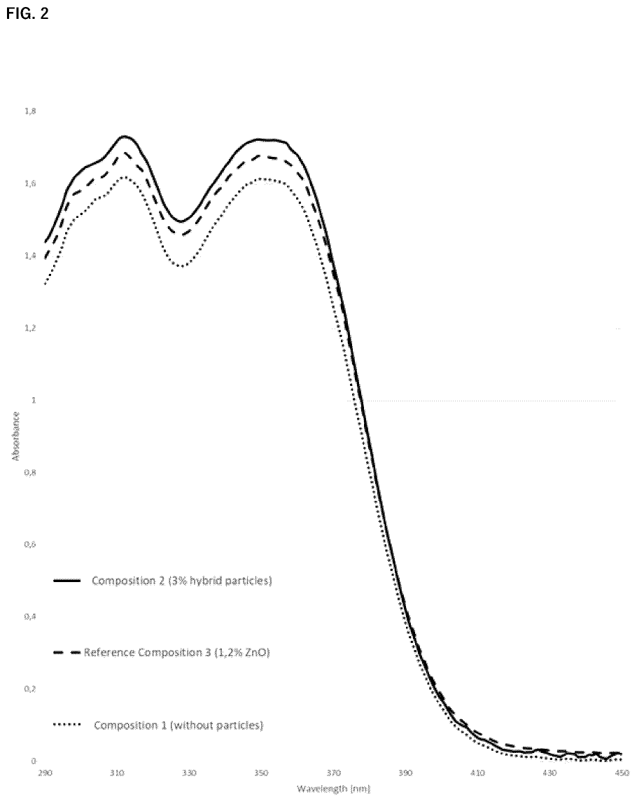

FIG. 2 shows the absorbance of the UV filter compositions 1, 2 and 3 in the range from 290 to 450 nm.

The composition 2 with 3 weight-% of added hybrid metal oxide particles shows increased absorption over the whole UV range from 290 to 450 nm. The in vitro SPF value of composition 2 showed an increment of 22% as compared to the placebo composition by the addition of 3 wt.-% of hybrid metal oxide particles according to example 1. Whereas, the in vitro SPF value of composition 3 containing 1.2% metal oxide particles (ZnO) only increased by 14% as compared to the placebo composition.

### Experiment 3: Transparency/Whitening Experiments

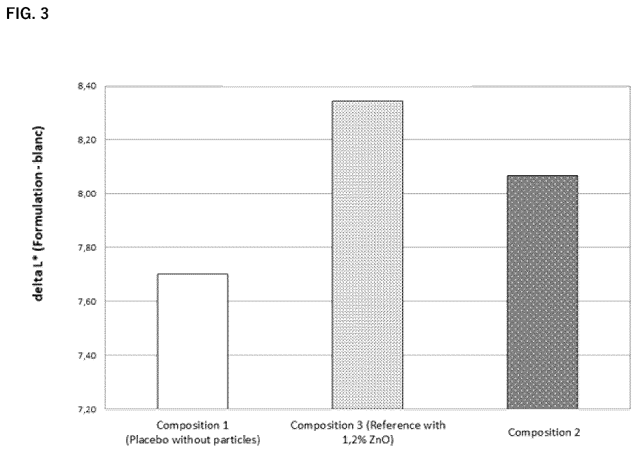

The whitening effect was determined for the compositions 1, 2 and 3 prepared in experiment 2 by color measurement as described above. The results for these compositions are summarized in FIG. 3.

FIG. 3 shows the difference of the lightness value L* of the UV filter compositions 1, 2 and 3. It was observed from FIG. 3 that Composition 2 (containing 3.0 wt. % of hybrid metal oxide particles) showed lower scattering of the visible light and thus produced a lower whitening effect on the skin.

On the contrary, Composition 3 (containing 1,2% ZnO) showed higher scattering of the visible light and thus produced a higher whitening effect on the skin. FIG. 3 shows that delta L* of Composition 3 increases to 8.3 whereas delta L* of composition 2 (3% hybrid metal oxide particles) only increases to 8.1.

Thus, the composition containing hybrid metal oxide particles of the presently claimed invention showed a lower increment in the delta L* value as compared to the composition with the same amount of a single metal oxide particles.

1.-38. (canceled)

39. A method for increasing the sun protection factor of a UV filter composition, the method comprising adding hybrid metal oxide particles to the UV filter composition,
the hybrid metal oxide particles comprising a continuous matrix of at least one first metal oxide having embedded therein an array of metal oxide particles, the metal oxide particles comprising at least one second metal oxide;
wherein the first metal oxide and the second metal oxide are independently selected from the group consisting of silica, titania, alumina, zirconia, ceria, iron oxides, zinc oxide, indium oxide, tin oxide, chromium oxide, and mixtures thereof.

40. The method according to claim 39, wherein the hybrid metal oxide particles are non-porous.

41. The method according to claim 39, wherein the amount of the hybrid metal oxide particles is in the range from 0.1 to 25.0 wt. % based on total weight of the UV filter composition.

42. The method according to claim 39, wherein the hybrid metal oxide particles have an average diameter in the range of 0.5 μm to 100.0 μm.

43. The method according to claim 39, wherein the amount of the metal oxide in the hybrid metal oxide particles is in the range of 60.0 to 100.0 wt. % based on total weight of the hybrid metal oxide particles.

44. The method according to claim 39, wherein the amount of the first metal oxide is in the range from 2 to 90 wt. % based on total weight of the hybrid metal oxide particles.

45. The method according to claim 39, wherein the first metal oxide is titania.

46. The method according to claim 39, wherein the amount of the second metal oxide is in the range of 10 to 98 wt. % based on total weight of the hybrid metal oxide particles.

47. The method according to claim 39, wherein the second metal oxide is silica.

48. The method according to claim 39, wherein the metal oxide particles comprising at least one second metal oxide have an average diameter in the range of 50 nm to 999 nm.

49. The method according to claim 39, wherein the weight ratio of the first metal oxide to the second metal oxide is in the range of 1:50 to 10:1.

50. The method according to claim 39, wherein the weight ratio of the first metal oxide to the second metal oxide is 2:3.

51. The method according to claim 39, wherein the continuous matrix further comprises at least one binder.

52. The method according to claim 52, wherein the binder is selected from silica, sodium silicate, magnesium silicate, calcium silicate, aluminum silicate, aluminum oxide hydroxide, sodium oxide, calcium carbonate, calcium aluminate, bentonite, kaolinite, montmorillonite, and combinations thereof.

53. The method according to claim 39, wherein the second metal oxide particles have a core-shell structure.

54. The method according to claim 39, wherein the array of the second metal oxide particles is an ordered array.

55. The method according to claim 39, wherein the array of the second metal oxide particles is a disordered array.

56. The method according to claim 39, wherein the second metal oxide particles have a surface functionalization.

57. The method according to claim 39, wherein the hybrid metal oxide particles comprise a surface functionalization.

58. The method according to claim 39, wherein the hybrid metal oxide particles exhibit color in the visible spectrum at a wavelength in the range of 380 nm to 800 nm.

59. The method according to claim 39, wherein the UV filter composition further comprises an UV absorber selected from the group consisting of
(d₁) p-aminobenzoic acid derivatives;
(d₂) salicylic acid derivatives;
(d₃) benzophenone derivatives;
(d₄) dibenzoylmethane derivatives;
(d₅) diphenyl acrylates;
(d₆) 3-imidazol-4-yl-acrylic acid and its esters;
(d₇) benzofuran derivatives;
(d₈) polymeric UV absorbers;
(d₉) cinnamic acid derivatives;
(d₁₀) camphor derivatives;
(d₁₁) hydroxyphenyltriazine derivatives;
(d₁₂) benzotriazole derivatives;
(d₁₃) trianilino-s-triazine derivatives;
(d₁₄) 2-phenylbenzimidazole-5-sulfonic acid and salts thereof;
(d₁₅) methyl o-aminobenzoates;
(d₁₆) homosalates;
(d₁₇) tris-biphenyltriazine derivatives;
(d₁₈) TiO₂, ZnO and mica;
(d₁₉) benzylidenemalonates;
(d₂₀) merocyanine derivatives;
(d₂₁) phenylene bis diphenyltriazines;
(d₂₂) imidazoline derivatives;
(d₂₃) diarylbutadiene derivatives;
(d₂₄) amino hydroxybenzoyl hexyl benzoate derivatives; and
(d₂₅) bis-(diethylamino hydroxybenzoyl benzoyl)-piperazine derivatives.

60. The method according to claim 39 for protecting the skin against ultraviolet radiations and high energy visible light.

61. The method according to claim 39, wherein the method further minimizes or masks the whitening effect of the UV filter composition and maintains its transparency.

62. A UV filter composition comprising the hybrid metal oxide particles in the range of 0.1 to 25.0 wt. % based on total weight of the UV filter composition,
the hybrid metal oxide particles comprising a continuous matrix of at least one first metal oxide having embedded therein an array of metal oxide particles, the metal oxide particles comprising at least one second metal oxide;
wherein the first and the second metal oxide is at least one independently selected from the group consisting of silica, titania, alumina, zirconia, ceria, iron oxides, zinc oxide, indium oxide, tin oxide, chromium oxide, and mixtures thereof.

63. The UV filter composition according to claim 62 wherein the hybrid metal oxide particles are non-porous.

64. The UV filter composition according to claim 62 comprising
a. water; and
b. the hybrid metal oxide particles in the range of 0.1 to 25.0 wt. % based on total weight of the UV filter composition.

65. The UV filter composition according to claim 62 comprising
a. water;
b. oil; and
c. the hybrid metal oxide particles in the range of 0.1 to 25.0 wt. % based on total weight of the UV filter composition.

66. The UV filter composition according to claim 62 comprising
a. oil; and
b. the hybrid metal oxide particles in the range of 0.1 to 25.0 wt. % based on total weight of the UV filter composition.

67. The UV filter composition according to claim 62, wherein the hybrid metal oxide particles have an average diameter in the range of 0.5 μm to 100.0 μm.

68. The UV filter composition according to claim 62, wherein the UV filter composition further comprises an UV absorber selected from the group consisting of
(d₁) p-aminobenzoic acid derivatives;
(d₂) salicylic acid derivatives;
(d₃) benzophenone derivatives;
(d₄) dibenzoylmethane derivatives;
(d₅) diphenyl acrylates;
(d₆) 3-imidazol-4-yl-acrylic acid and its esters;
(d₇) benzofuran derivatives;
(d₈) polymeric UV absorbers;
(d₉) cinnamic acid derivatives;
(d₁₀) camphor derivatives;
(d₁₁) hydroxyphenyltriazine derivatives;
(d₁₂) benzotriazole derivatives;
(d₁₃) trianilino-s-triazine derivatives;
(d₁₄) 2-phenylbenzimidazole-5-sulfonic acid and salts thereof;
(d₁₅) methyl o-aminobenzoates;
(d₁₆) homosalates;
(d₁₇) tris-biphenyltriazine derivatives;
(d₁₈) TiO₂, ZnO and mica;
(d₁₉) benzylidenemalonates;
(d₂₀) merocyanine derivatives;
(d₂₁) phenylene bis diphenyltriazines;
(d₂₂) imidazoline derivatives;
(d₂₃) diarylbutadiene derivatives;
(d₂₄) amino hydroxybenzoyl hexyl benzoate derivatives; and
(d₂₅) bis-(diethylamino hydroxybenzoyl benzoyl)-piperazine derivatives.

69. The UV filter composition according to claim 62, further comprising at least one emulsifier in the range of 1.0 to 20.0 wt. % based on total weight of the UV filter composition.

70. The UV filter composition according to claim 69, wherein the emulsifier is selected from the group consisting of an anionic emulsifier, a cationic emulsifier, a nonionic emulsifier, and a polymeric emulsifier.

71. The UV filter composition according to claim 62, further comprises additives selected from the group consisting of thickener, active ingredients, preservatives, and perfumes.

72. The UV filter composition according to claim 62, wherein the UV filter composition is a sunscreen composition.

73. The UV filter composition according to claim 62, wherein the UV filter composition is day care composition.

74. The UV filter composition according to claim 62, wherein the UV filter composition is at least one selected from cream, emulsion, milk, lotion, ointment, oils, gels, sprays, aerosols, daily care lotion, water resistant emulsion, oil in water emulsion, oil in water lotion.
